(19) United States
(12) Patent Application Publication
(10) Pub. No.: US 2012/0277411 A1
(43) Pub. Date: Nov. 1, 2012

(54) SINGLE CHAIN ANTIBODIES FOR PHOTOSYNTHETIC MICROORGANISMS AND METHODS OF USE

(76) Inventors: George A. Oyler, Lincoln, NE (US); Julian N. Rosenberg, Naugatuck, CT (US); Donald P. Weeks, Lincoln, NE (US)

(21) Appl. No.: 13/441,951

(22) Filed: Apr. 9, 2012

Related U.S. Application Data
(60) Provisional application No. 61/472,750, filed on Apr. 7, 2011.

(51) Int. Cl. C07K 14/405; C07K 1/14; C07K 19/00

(52) U.S. Cl. 530/387.3

(57) ABSTRACT

A single chain antibody that binds algae is described. The single chain antibody for algae is used to capture algae onto bioactive films. The single chain antibody is also used in a chimeric construct having a substrate binding domain and a single chain antibody domain. Dimers, trimmers, and multimer constructs are also described that aid in collection of algae from liquid mixtures by causing flocculation of algae cells.

### CROSS-REFERENCE TO RELATED APPLICATIONS

This application claims priority under 35 U.S.C. §119(e) from U.S. Provisional Application Ser. No. 61/472,750, entitled Single Chain Antibodies for Photosynthetic Microorganisms and Method of Use, filed on Apr. 7, 2011, the specification of which is incorporated herein by reference in its entirety.

### STATEMENT OF GOVERNMENT RIGHTS

This invention was made with government support under Grant No. DE-EE0003373 awarded by the United States Department of Energy. The government has certain rights in the invention.

### BACKGROUND OF THE INVENTION

1. Field of the Invention

The present invention relates to single chain antibodies for photosynthetic microorganisms.

2. Background of the Prior Art

Some of the most challenging aspects of large-scale microalgal cultivation include species management, control of contamination, immense water usage, and energy requirements to harvest biomass from relatively dilute solutions (e.g., 2 g algae per L water). In order to address these issues, novel microalgal cultivation systems require mechanisms of reducing water requirements, which in turn, facilitate harvesting. The ability to target the binding of a specific desired microalgal species represents a novel approach that provides particular advantages for growth and harvesting of high-value algal biomass with less or no contaminating organisms.

### SUMMARY OF THE INVENTION

One object of the present invention is to provide isolated single chain antibodies (VHH domains), which recognize photosynthetic microorganism cells. The isolated single chain antibodies are capable of binding a photosynthetic microorganism, such as algae. A second object of the present invention to provide a method for isolating single chain antibodies specific for algae, the method comprising the steps of immunizing an animal in the camelid family with an algae strain, collecting blood sample from the animal; preparing a cDNA library from the blood sample; expressing the cDNA library on a phage; panning the phage for antibodies specific to live algae strains; and isolating the antibodies identified in the panning step. The panning step of the method comprises the following steps obtaining a pellet of live algae; adding blocking reagents to the pellet; exposing the phage to the pellet; and eluting the phage.

A further object of this invention is to provide single chain antibodies that may be used in various applications. In one embodiment, a chimeric fusion peptide construct, comprising an isolated single chain antibody domain capable of binding an alga cell, and a substrate binding domain is provided. The peptide construct is capable of binding algae cells such as Chlamydomonas reinhardtii, Chlorella variabilis, Coccomyxa sp., Nannochloropsis oceanica, and Thalassiosira pseudonana.

In one example, the chimeric fusion construct has a single chain antibody and a substrate-binding domain. The chimeric fusion construct attaches to a substrate that is the substrate for the substrate binding domain, such as cellulose, creating a functionalized substrate. The functionalized substrate is then utilized to capture photosynthetic microorganisms that bind to the single chain antibody segment (VHH domain) of the chimeric fusion construct.

In one aspect of the invention, the isolated single chain antibodies are used to induce flocculation of the microorganism, e.g., algae, in a liquid suspension. A method for collecting algae from an algae culture, the method comprising the steps of introducing a single chain antibody specific for algae to the algae culture causing the algae to flocculate and collecting the algae after flocculation. In yet another embodiment, the single chain antibody is introduced by a chimeric fusion peptide construct comprising at least two copies of the single chain antibody. In another embodiment, the chimeric fusion peptide construct comprises a first single chain antibody specific for a first algae species, and a second single chain antibody specific to a second algae species. In such method, the first algae species is a viral host to a virus that causes lysis of the second algae species.

### BRIEF DESCRIPTION OF THE DRAWINGS

The numerous advantages of the present invention may be better understood by those skilled in the art by reference to the accompanying figures in which:

FIG. 1 is a graphical representation of a comparison of the sequences of a single chain antibody specific for Chlamydomonas reinhardtii UTEX 2244 isolated from Desi antisera.

FIG. 2 is a schematic representation of a various chimeric constructs using single chain antibodies.

FIG. 3 is a schematic representation of a functionalized substrate with attached constructs having a single chain antibody VHH domain and a binding domain. The schematic also shows algae attached to the substrate by the single chain antibody construct. In addition, the schematic shows algae flocculation caused by the presence of single chain antibody dimers.

FIG. 4 is a schematic representation of flocculation of algae instigated by single chain antibody dimers.

FIG. 5 is a schematic representation of algae flocculation facilitated by surface display of single chain antibodies on the cell surface of microorganisms.

FIG. 6 is a schematic representation of simultaneous target algae flocculation and lysis by proximity to Chlorella variabilis NC64A viral infection facilitated with bivalent single chain antibody fusions.

FIG. 7 is a sample graph of the serum titers showing specificity for Chlamydomonas reinhardtii UTEX 2244.

FIG. 8 is a pair of Western blots using antisera against Nannochloropsis oceanica OZ-1, Chlorella variabilis NC64A, Coccomyxa sp. C-169, Thalassiosira pseudonana CMMP 1335, and Chlamydomonas reinhardtii UTEX 2244, in accordance with one embodiment of the present invention.

FIG. 9 is a commassie blue stained gel of purified single chain antibodies specific to Chlorella spp.

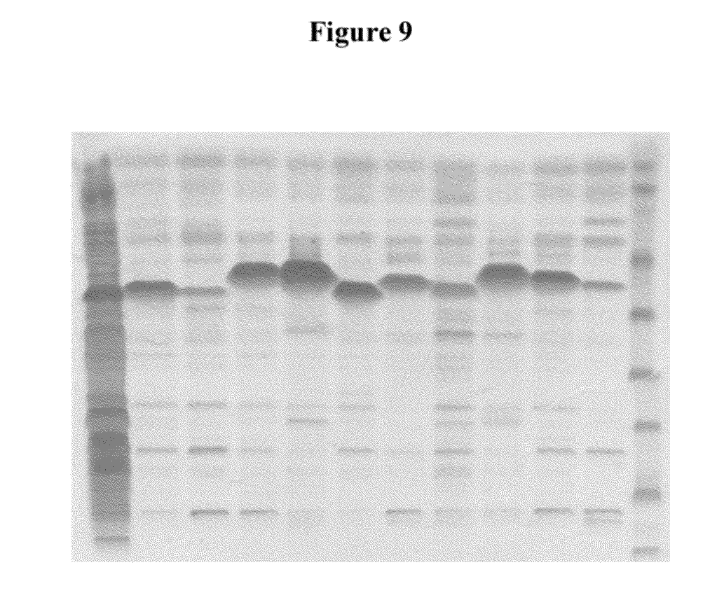

FIG. 10 is a confocal image of Chlamydomonas reinhardtii (cc124) treated with a construct having the fluorescent marker mCherry.

FIG. 11 is a confocal image of Chlamydomonas reinhardtii (cc124) treated with constructs comprising the GFP and mCherry tags.

FIG. 12 shows a schematic of a construct in accordance with one embodiment of the present invention that comprises a substrate binding domain and a single chain antibody for algae.

FIG. 13 is a picture that shows different cell density of Chlamydomonas reinhardtii cells in a). culture was inoculated by a piece of Whatman paper pretreated with CBD-mCherry-VHH(JGJ-B11) protein and followed by incubated with living C. reinhardtii cells and b). the same treatment but pretreated with CBD-mCherry control protein. Image was taken three days post inoculation.

### DETAILED DESCRIPTION

The invention summarized above may be better understood by referring to the following description, accompanying drawings and claims. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

As described in this application a “single chain antibody”, “nanobody”, or “VHH” is a peptide that is capable of binding to specific substrates. These peptides are also called “camelid” antibodies because they are generally derived from members of the camelid family (e.g., camels, llamas, alpacas), they are also found in cartilaginous fish (e.g., sharks). Typically, the VHH size range from 10 kDa to 14 kDa, with many in the 12 kDa range. It is contemplated, however, that the size of the single chain antibodies is not material to their function and single chain antibodies may be larger or smaller, provided they are capable of binding their substrates. The single chain antibodies disclosed herein are specific for photosynthetic microorganisms, such as algae, diatoms, cyanobacteria and others. Single chain antibodies correspond to the variable antigen-binding region of the camelid family heavy chain-only antibodies. Single chain antibodies, have remarkable properties such as subnanomolar affinity, rapid refolding after denaturation, extremely reliable, robust, soluble expression in nearly all protein expression platforms (e.g., E. coli) and ability to screen for desired single chain antibodies by phage display in a high through-put manner (Saerens et al. 2008; Arbabi-Ghahroudi et al. 2005; Muyldermans et al. 2001). The antibody domains disclosed herein have high affinity for surface-exposed cell wall protein(s) and other molecules of the microalgal species of interest.

As used in this application “amino acid sequence identity” means percent similarity of amino acid residues between two sequences. For example, a sequence that has an amino acid sequence identity of 85% with another sequence means that the amino acid sequence in question has 85% of the same amino acid residues of the amino acid sequence to which it is being compared. Two amino acid sequences are said to be “identical” if the two sequences, when aligned with each other, are exactly the same with no gaps, substitutions, insertions or deletions. Two amino acid sequences are defined as being “substantially identical” if, when aligned with each other, (i) no more than 30%, preferably 20%, most preferably 15% or 10%, of the identities of the amino acid residues vary between the two sequences; (ii) the number of gaps between or insertions in, deletions of and substitutions of, is no more than 10%, preferably 5%, of the number of amino acid residues that occur over the length of the shortest of two aligned sequences; or (iii) have only conservative amino acid substitutions (in one polypeptide as compared to another) that do not significantly affect the folding or activity of the polypeptide. The entire amino acid sequence of two proteins may be substantially identical to one another, or sequences within proteins may demonstrate identity or substantial identity with sequences of similar length in other proteins. In either case, such proteins are substantially identical to each other. Typically, stretches of identical or substantially identical sequences occur over 5 to 25, preferably 6 to 15, and most preferably 7 to 10, nucleotides or amino acids. See e.g., United

States Patent Publication Serial Number 2003/0161809.

The nucleotide sequences provided herein are only examples of some of the representative sequences that code for a specific peptide sequence. A person of ordinary skill in the art would understand that standard computer methods can be utilized to determine the nucleotide sequence of a given amino acid chain, peptide, or polypeptide. Thus, the nucleotide sequences contained herein are examples of the coding sequences for the single chain antibodies of the present invention and that other nucleotide sequences that code for the same peptides are within the scope of the present invention.

The term “algae” as used in this application refers to a wide range of photosynthetic microorganisms, which include eukaryotic green algae, diatoms, and cyanobacteria. Examples of algae genera include Chlorella, Botryococcus, Neochloris Auxenochlorella, Chlamydomonas, Dunaliella, Haematococcus, Coccomyxa, Schizochytrium, Crypthecodinium, Isochrysis, and Tetraselmis. Examples of diatom genera Phaeodactylum, Chaetoceros, Skeletonema, Thalassiosira, Nitzschia, Navicula. Examples of cyanobacteria genera include Anabaena, Synechococcus, Spirulina.

The term “substrate” as referred to in this application means a membrane or other solid or semi-solid material to which a single chain antibody can be attached. Some examples of substrates include cellulose membranes, nitrocellulose membranes, nylon membranes, hydrogels, alginates, carrageenans. The substrate, in some instances, is also referred to as a “film” or “biofilm”. Lau P S, Tam N F Y, Wong Y S (1997) Wastewater nutrients (N and P) removal by carrageenan and alginate immobilized Chlorella vulgaris. Environ Technol 18:945-951; Lau P S, Tam N F Y, Wong Y S (1998) Operational optimization of batchwise nutrient removal from wastewater by carrageenan immobilized Chlorella vulgaris. Water Sci Technol 38:185-192; Mallick N (2002) Biotechnological potential of immobilized algae for wastewater N, P and metal removal: a review. Biometals 15:377-390; Mehta S K, Gaur J P (2005) Use of algae for removing heavy metal ions from wastewater: progress and prospect s. CRC Rev Biotechnol 25:113-152; Shi J, Podola B, & Melkonian M (2007) Removal of nitrogen and phosphorus from wastewater using microalgae immobilized on twin layers: an experimental study J Appl Phycol 19:417-423; Munoz R, Kollner C, Guieysse B (2009) Biofilm photobioreactors for the treatment of industrial wastewaters Journal of Hazardous Materials 161: 29-34.

Single Chain Antibodies Specific for Algae

In one embodiment of the present invention, isolated single chain antibodies capable of binding algae cells are provided. The isolated single chain antibody, in accordance with one embodiment of the present invention, is capable of binding Chlamydomonas reinhardtii UTEX 2244, Chlorella variabilis NC64A, Chlorella sorokiniana CCTCC M209220, Coccomyxa sp. C-169, Nannochloropsis oceanica OZ-1, and Thalassiosira pseudonana CMMP 1335, among others. An isolated single chain antibody may bind one or more of the species listed above. In one embodiment of the present invention, several examples of single chain antibodies capable of binding Chlamydomonas reinhardtii are provided having amino acid SEQ ID Nos. 1 through 9 and nucleotide SEQ ID Nos. 10 through 18. In a further embodiment of the present invention, several examples of single chain antibodies capable of binding Chlorella variabilis are provided having amino acid SEQ ID Nos. 19 through 27 and nucleotide SEQ ID Nos. 28 through 36. In another embodiment of the present invention, several examples of single chain antibodies capable of binding Nannochloropsis oceanica are provided having amino acid SEQ ID Nos. 37 through 51 and nucleotide SEQ ID Nos. 52 through 66. In another embodiment of the present invention, several examples of single chain antibodies capable of binding Thalassiosira pseudonana are provided having amino acid SEQ ID Nos. 67 through 69 and nucleotide SEQ ID Nos. 70 through 72. The number of species described above show that single chain antibodies are capable of binding all types of algae.

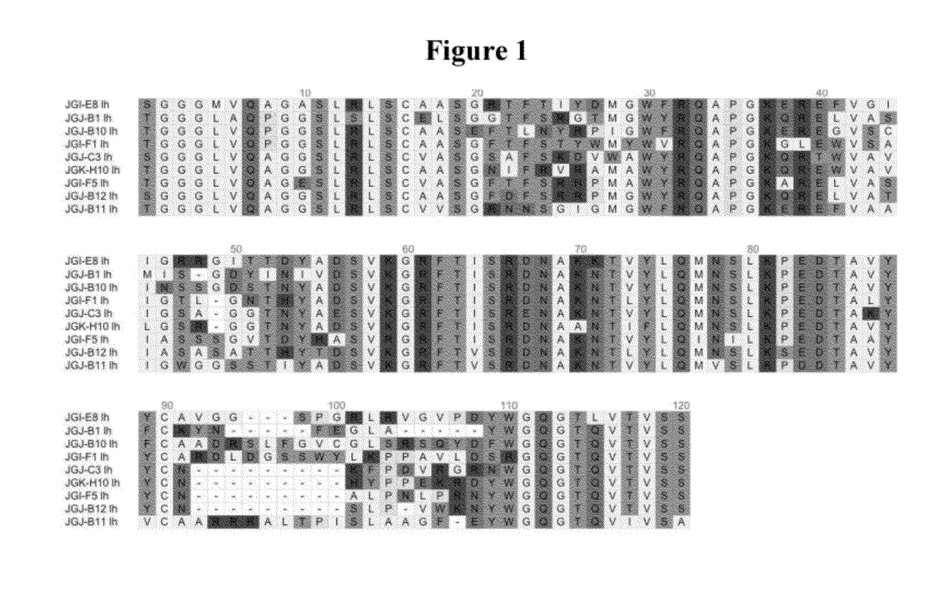

As shown in FIG. 1, single chain antibodies having sequence identity of at least 40% with the other isolated single chain antibodies described in this application are capable of binding algae. In some embodiments of the present invention, the single chain antibodies that have an amino acid sequence identity of at least 85%, preferably 85% to 90%, more preferably 90% to 95%, or which are “substantially identical” to the sequences provided herein are capable of binding algae.

Method of Isolating Single Chain Antibodies Specific for Algae

In one embodiment of the present invention, a method of isolating single chain antibodies is provided, as shown below:

1. Immunization: A camelid animal is immunized with the species of algae of interest. The animal can be immunized with live algae, dead algae, or algae membranes, proteins, and other molecules. The animal is exposed to the algae through injection or any other means known to a person of ordinary skill in the art to elicit an immune system response. In the example described below, two alpacas were immunized with four different species of killed algae every two or three weeks for up to six immunizations as needed to achieve acceptable titers of anti-algal surface antibodies within the serum.

2. Validation: Collect up to 10 ml of serum from each animal prior to initiation of algae immunization (i.e., preimmune sera) and approximately a week subsequent to each immunization. Validate immune response through Western Blot and ELISA analysis.

3. Prepare cDNA Library: After immune response has been validated, collect lymphocytes from the animals and prepare cDNA library. In accordance with one embodiment, the cDNA library was prepared from peripheral blood cell mRNA obtained approximately one week following the final immunization of each animal. The mRNA and cDNA libraries were prepared using the method described in Maass et al., 2007.

4. Phage Display: The cDNA library is then utilized to further isolate single chain antibodies specific for algae. In one exemplary embodiment, an M13 phage display library was prepared consisting of at least 1,000,000 independent transformants containing alpaca VHH coding DNA prepared from the cDNA obtained in step 3. The phage display library was prepared using the method described in Maass et al., 2007.

5. Identify VHHs specific for each algae species by panning: One phage pool is obtained following at least three panning cycles of selection to identify VHH proteins able to bind to the surface of each of the four different algae species used in the immunization. Each phage pool is separately selected for each of the four algae species. The modified panning method described in (Maass et al., 2007) are used. The modified panning methods comprises the following steps: First, collect a pellet of live microalgal cells by centrifugation, resulting in a ˜75 μl pellet for use as panning material. Next, block the cell surface using a solution of 4% non-fat milk in phosphate buffered saline with 0.1% Tween (PBST) by washing the cells in 1 ml milk-PBST (blocking solution), vortexing and centrifuging (3 min, 22° C., 3000×g). Repeat three times. Next, add 1 ml of fresh blocking solution and incubate for 30 min, rotating at room temperature. Finally, centrifuge (4 min, 22° C., 3000×g) to remove blocking solution. Primary 1° panning begins by adding 500 μl blocking solution and 500 μl of the master phage library to this material and rotating for 1 hr at room temperature in a microfuge tube. Then, washing 6 times in 1 ml PBST and rotating the final wash for 10 minutes at room temperature and eluting the phage pool as described below. The secondary pan (2°) follows a similar procedure, using 600 μl blocking solution and 400 μl of the phage from 1° elution, rotating for 1 hr at room temperature. Then, washing 10 times in 1 ml PBST and rotating the final wash for 10 minutes at room temperature and eluting the phage pool as described below. The tertiary pan (3°) is the most stringent and proceeds with 50 ml of blocking solution and only 5 μl of phage from 2° elution, rotating for 10 min at room temperature. The washing steps include a total of 10 times in PSBT using the following volumes: 1 time in 50 ml, 4 times in 14 ml, and 5 times in 1 ml. Rotate the final wash for 10 minutes at room temperature and eluting the phage pool as described below.

6. Elution of Phage: After the final wash of the panning step, centrifuge (3 min, 22° C., 3000×g); save the supernatant as the 1°, 2°, or 3° wash and the remaining algae pellet contains phage that have adhered to the cell surface. Add 500 μl 0.2 M glycine pH 2.2 to this algae pellet and resuspend by vortexing. Rotate for 10 min at room temperature; collect the algae pellet by centrifugation. Transfer the supernatant to new microfuge tube containing 75 μl 1 M Tris pH 9.1. Pipette up and down immediately to neutralize the eluate. Wash algae pellet 2 times in 1 ml PBS (vortex; spin 3 min, 22° C., 3000×g), remove wash. Add 500 μl of E. coli ER2738 overnight culture, vortex, incubate at 37° C. for 15 min. Then, briefly centrifuge to collect all liquid that may adhere to the cap. Add the algae and E. coli ER2738 mixture to the first (already neutralized) eluate. Use this combined, and final eluate to titer in the 10²-10⁸ range of serial dilution.

7. Identify ‘best’, unique VHH clones: In one embodiment of the present invention, the “best” VHH clones are defined as having different BstN1 and HaeIII fingerprints and the strongest signal using phage ELISAs on microtiter plates coated with algae extracts from each of an algae species of interest. Each of these clones produces a single chain antibody specific for a particular algae strain.

8. Identify ‘best’, unique VHH clones: In one embodiment of the present invention, the “best” VHH clones are defined as having different BstN1 and HaeIII fingerprints and the strongest signal using phage ELISAs on microtiter plates coated with algae extracts from each of an algae species of interest. Each of these clones produces a single chain antibody specific for a particular algae strain.

Methods of Using Single Chain Antibodies

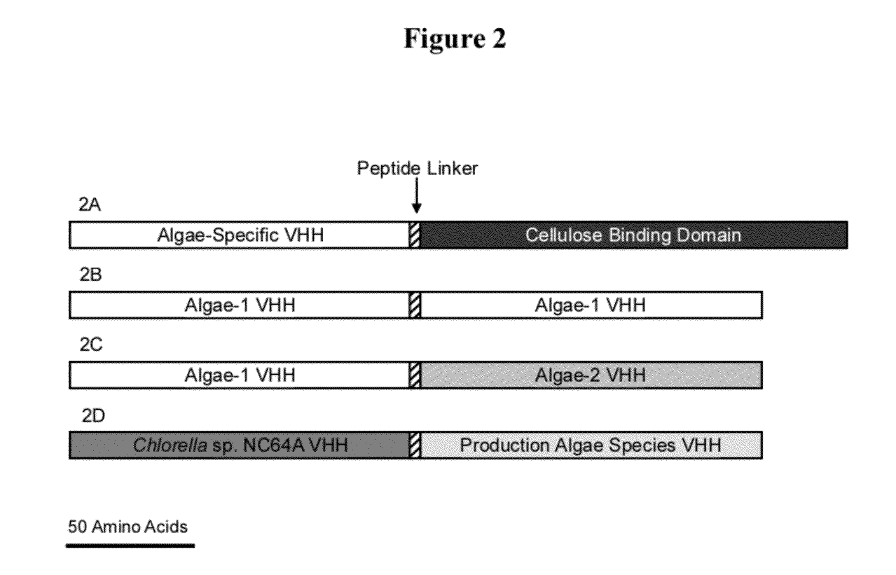

The single chain antibodies isolated in accordance with the method described above have practical applications as more fully described below. As shown on FIG. 2, single chain antibodies combined with substrate binding domains to capture algae on the substrate to which the substrate binding domain attaches (FIG. 2A). Single chain antibody dimers, trimmers, and multimers specific for one algae species are used for cultivation and collection of algae through flocculation (FIG. 2B). Single chain antibodies for one species combined with single chain antibodies from a different species, are also used for flocculation and cultivation of algae (FIG. 2C and 2D).

a. Chimeric Fusion Peptides for Algae Immobilization in Solid or Semi-Solid Substrates

In one embodiment, the single chain antibodies are part of a chimeric fusion peptide construct as shown in FIG. 2A. A chimeric fusion protein construct has an isolated single chain antibody fused to a substrate binding domain. The substrate binding domain allows the chimeric fusion protein to bind materials used for various purposes. In a further embodiment of the present invention, the chimeric fusion protein construct has a linker between the single chain antibody and the substrate binding domain. The linker has a length of 5 to 50 amino acids, preferably 10 to 40, more preferably 20 to 30 amino acids. One example is the (EAAAR)ₙ linker domain (e.g., Yan et al., 2007), where n is the number of repeats of the EAAAR sequence ranging between n=1 to 50 repeats. A linker, in accordance with one embodiment, has at least one repeat of the EAAAR sequence. Other linkers contemplated within the scope of the present invention will include multiple repeats of the linker sequence. Another linker sequence is amino acids 1 to 206 of human SNAP25a with the cysteine residues mutated to serine or amino acids 140 to 206 of SNAP25 as a linker. More generally linker sequences between 5 and 500 amino acids which assume a nonstructured configuration and/or extended rod configuration may be used.

The substrate domain of one construct is a cellulose binding domain that allows the chimeric fusion protein to bind to cellulose while the single chain antibody is utilized to capture algae cells, algae cell fragments, or other antigens for which it has specificity. Other examples of substrate binding domains include: glutenin binding domain, lectin binding domain, and fibronectin binding domain. See e.g. Pankov R, Yamada K M (2002) Fibronectin at a glance. J Cell Sci. 115:3861-3863; Weegels P L, Hamer R J, Schofield I D Functional properties of wheat glutenin. (1996) Journal of Cereal Science, 23:1-18; Komath S S, Kavitha M, Swamy M J (2006) Beyond carbohydrate binding: new directions in plant lectin research Org Biomol Chem. 4:973-988; Lynd L R, Weimer P J, van Zyl W H, and Pretorius I S (2002) Microbial Cellulose Utilization: Fundamentals and Biotechnology Microbiol. Mol. Biol. Rev. 66: 506-577; Linder M, Salovuori I, Ruohonen L, Teeri T T (1996) Characterization of a Double Cellulose-binding Domain JBC 271:21268-21272. There are many such elements well defined in biotechnology particularly from cellulosome research, which have been expressed recombinantly as approximately 8 kDa peptides. They are functional, fold well, are easily expressed, and their sequences are well-known. (Shoham et al. 1999; Fierobe et al. 2001; Kakiuchi et al. 1998; Lamed et al. 1983). The cellulose binding domain is one of the preferred domains because it allows the construct to bind to cellulose based substrates, which are abundant, inexpensive, biodegradable and easy to utilize. In yet a further embodiment of the present invention, the single chain antibodies are covalently attached to a substrate or membrane.

In a further embodiment of the present invention, the substrate is a selective membrane biofilm for immobilizing algae for biofuel production after growing the algal cell biomass in liquid culture. The selective substrate or biofilm has a thickness in the range of 0.1 mm to 1.0 cm, more preferably approximately 1 mm.

Microalgae are poised to be a mainstay of sustainable bioprocessing as a versatile photosynthetic production platform for pharmaceuticals, nutritionals, commodities, and biofuels. The single chain antibodies of the present invention provide a method for the cultivation of algae that allows for regulation of biomass composition, maintains culture integrity, and improves harvesting efficiency. In the first step of the method, algae are immobilized on a substrate. This immobilization step is accomplished by using the chimeric fusion protein described above to attach the single chain antibody to the substrate and the single chain antibody to capture the algae. In the second step of the process, algae are grown on the substrate by providing the required nutrients and continuously moving water over the substrate loaded with algae.

b. System for Cultivating Algae on Solid Substrates

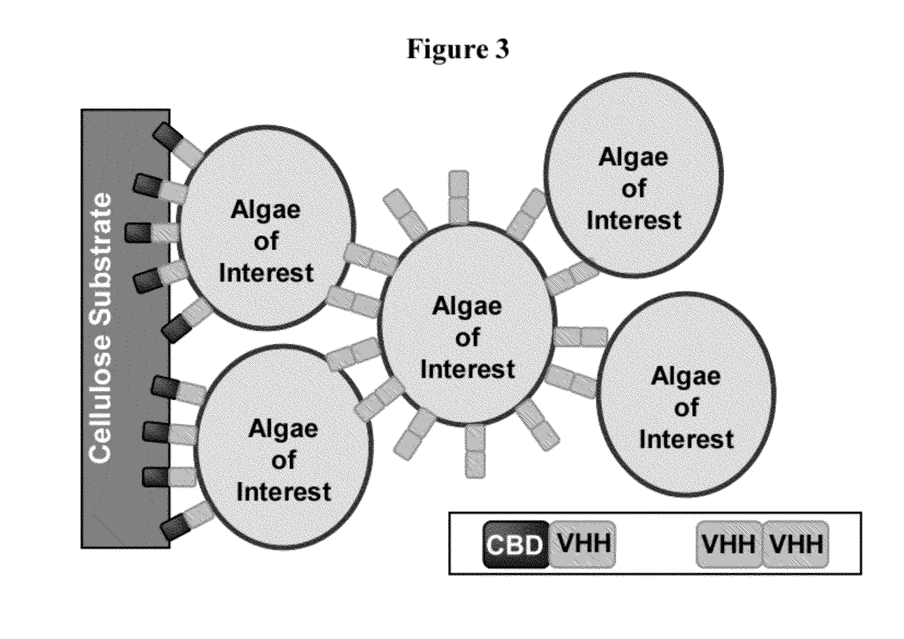

The method described above, is facilitated through a system for cultivating algae as shown on FIG. 3. The system includes a substrate that has the ability to bind a chimeric peptide construct having a single chain antibody that binds algae and a substrate binding domain.

In one embodiment of the present invention the substrate has functional groups on its surface that are recognized by the substrate binding domain. A second component of the system is the chimeric peptide construct capable of binding algae as described above.

In some embodiments of the present invention, a third element of the system is a strain of algae for cultivation. In an alternative embodiment, an additional element for the system is a chimeric peptide substrate comprising a dimer, trimer, or multimer of single chain antibodies specific to a particular algae species. The chimeric peptide comprising at least two single chain antibodies allows the aggregation/flocculation of algae to algae that is bound to the solid or semi-solid substrate. Another element of the system is a water source, such as a water reservoir, and channels to direct water to the substrate. Yet a further element of the system is source of nutrients, which may be the water source to which nutrients can be added. In yet a further embodiment, the system is provided in a kit, which consists of a package containing at least two of the elements of the system. The system having a substrate with the construct attached to it through the binding domain is utilized to capture organisms that the single chain antibodies recognize and bind.

Unlike the conventional method of cultivating algae biomass as a suspension in a large volume of liquid medium, which must be constantly circulated within open ponds or closed photo bioreactors, the substrate-based system in accordance with one embodiment of the present invention takes advantage of immobilized algal cell growth on the surface of a biodegradable substrate with a minimal amount of water to be continuously passed over the surface. In this system production algae are tethered to the substrate and covered with a thin film of liquid. The system provides significant advantages over existing photobioreactor systems. In existing bioreactor systems it is difficult to harvest algae from dilute solutions. In the system disclosed herein, the algae are already attached to a substrate surface and, thus, harvesting the organisms is not complicated by the low density of algae in the liquid media. The system described herein utilizes less water and carbon dioxide delivery to a thin substrate is vastly improved. Furthermore, interorganism shading and mixing problems are minimized due to the shallow presence of liquids in the system.

The current system improves optimal algae growth over existing systems. An aerial productivity of 25 to 30 g/m²/day is at or near the limit of current productivity for a raceway pond operated at optimal growth over a period of months. In the system described herein, an aerial productivity of 75 to 100 g/m²/day—well outside the range of any currently existing industrial scale algal growth system—can be achieved by a growth of an algae layer of 75-100 μm thick over a 1 m² cellulose substrate.

One application of this novel growth system is to maintain the desired algal organism on the solid growth substrate despite the presence of a surrounding polymicrobial community or contaminants. In one embodiment of the present invention, the substrate bound algae can be utilized for the recovery of nitrogen and phosphorus from nutrient-rich effluent from municipal or agricultural wastewater sources (Muiloza et al. 2009). In doing so, these vital nutrients will not only be sequestered from damaging environmental release, but also sustain the biosynthesis of useful products from algae while simultaneously reducing operating costs of the system.

In a further embodiment, the immobilized algae can be used for the production of biofuels and other products (Rosenberg et al. 2008). In one exemplary embodiment, the desired products may be obtained by removing the substrate covered with algae engineered to produce the desired byproduct and extracting the product through standard methods. In one example, the algae cells are lysed and the products are subsequently collected. In another exemplary embodiment, algae that are engineered to secrete biofuel or other products, such as medium chain fatty acids, can be utilized. (Liva et al. 2010; Reppas & Ridley 2010). In other examples, the algae species secretes the desired product such as volatile metabolites including isoprene and monoterpenes. Isoprene secreting Chlamydomonas are currently available (Bentley and Melis, 2011 and Lindberg et al, 2010). The algae on the substrate convert sunlight into secreted products, such as lipids. Other algae secrete sugars such as glucose, sucrose, and maltose. Transport of these molecules is facilitated by transporters such those coded by the SWEET genes and ABC type transporters. Secretion of the products onto the water stream makes it much easier to collect such products.

Another advantage of the system described above is that alga of interest are adsorbed to the selective surface while all other potential contaminant organisms are washed away along with the flow of liquid. Based on the same principle, other morphologies are certainly conceivable. The substrate may be a fine mesh to increase the surface area, a screen through which water may pass, or a multiplicity of fibers. In one aspect of the present invention, the mesh or multiplicity of fibers is functionalized with single chain antibodies as described above. Essentially any form of single chain antibody functionalized substrate may be used to collect the algae (e.g., wood chips, scrap paper) such that the algae-coated material can be settled or otherwise collected from solution.

In one preferred embodiment, the system consist of a very thin (˜1 mm) membrane, a small gas space (˜1 cm) and an upper and lower clear film membrane for containment. The biofilm membrane or substrate is self-contained, which prevents evaporative losses. When used in large scale, the gas circulation of the biofilm or membrane is passed through ground heat pump cooling systems (10° C.) to condense the water vapor and maintain the appropriate biofilm/substrate temperature. Areas close to an ocean could also use the lower depths as a heat sink and water condensation for the gas circulation from the biofilm.

c. Single Chain Antibodies Used for Cultivation of Algae Through Flocculation

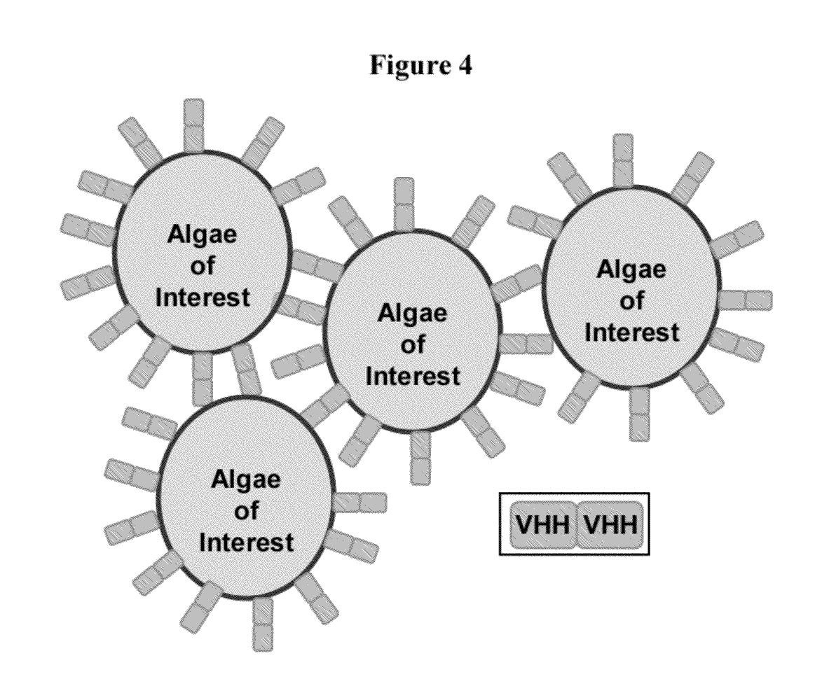

Flocculation refers to the ability to cause algae cells to aggregate and sink to the bottom of a water reservoir. In one embodiment of the present invention, the single chain antibodies are expressed in the surface of algae causing flocculation of algae cells for easy harvest as shown in FIGS. 4, 5, and 6. Surface display of single chain antibodies may be accomplished, by way of example, using the method of Boder and Wittrup (1997). In yet a further embodiment, flocculation of algae is achieved through the use of chimeric peptide constructs, as described above, where the single chain antibody is linked to a substrate binding domain that recognizes other algae cells. The substrate binding domain in these types of constructs may include other single chain antibodies capable of binding the same species of algae or a different species. Such substrate binding domains fix the single chain antibodies to specific substrates, which in turn are utilized for collecting algae or other target microorganism recognized by the single chain antibodies. These and other applications are explained in further detail below.

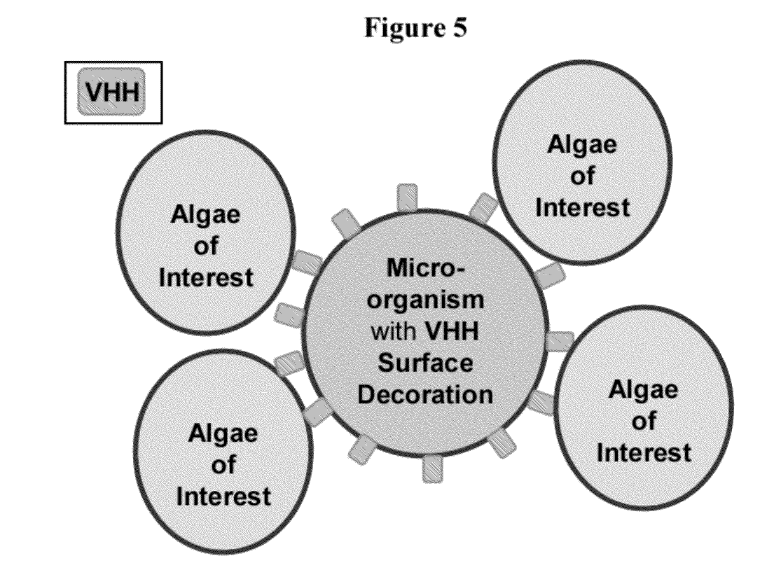

Single chain antibodies, dimers, trimers, or multiple-copy constructs of the single chain antibodies are used to induce flocculation of the microorganisms by adding the constructs to the liquid solution in which the organisms are growing. Constructs that include dimers or trimers of VHH domains for one or more algae may be added to an algal culture to instigate flocculation as shown in FIG. 4. The algae mass can then be recovered by low speed centrifugation. In a further embodiment, the VHH domains are displayed on the surface of yeast, diatoms, or even the algae production strain of interest as shown in FIG. 5. The organisms displaying the single chain antibodies on their surface will collect the algae as described above.

d. Single Chain Antibodies Used for Flocculation of Different Algae Species for Specific Purposes.

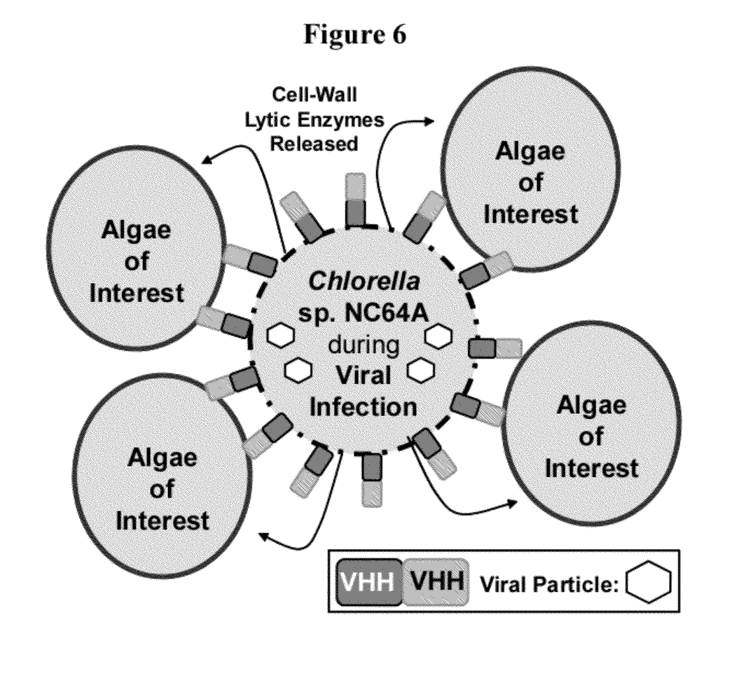

In yet a further embodiment, two production strains are coupled for harvest. If microalga-1 is engineered to display VHH domains for microalga-2 on its surface and microalga-2 exhibits similar affinity for microalga-1 by VHH display, the two algae are grown separately and then mixed together in order to harvest both organisms rapidly. In one additional example, these algae single chain antibodies are used to take advantage of other unique proximity relationships that may facilitate harvesting and lysis of microalgal cells as shown in FIG. 6.

In another embodiment, a strain of algae infected with a virus that causes lysis of another strain of algae is used to facilitate collection of the byproduct of a production strain of algae. In one example, the lytic strain is engineered to express the single chain antibody specific for the production strain. When the two strains are mixed, the single chain antibody expressed in the surface of the lytic strain causes flocculation of the lytic strain with the production strain. The virus on the lytic strain causes degradation of the production strain releasing the production strain's products. Alternatively, the production strain and a lytic strain that does not express the single chain antibodies are mixed with a chimeric peptide construct that is capable of binding both strains as explained above.

For example, the alga Chlorella variabilis NC64A is host to various Chlorella viruses (Sugimoto et al. 2000). Upon viral infection, lytic enzymes are expressed and cause the breakdown the alga's cell wall during the viral life cycle. These lytic enzymes act as an important tool to deconstruct the cell wall components of other microalgae; thus, allowing release of intracellular oils and other coproducts for facilitated recovery. The use of single chain antibodies to bring production strains of algae in direct contact with C. variabilis NC64A during viral infection promotes efficient lysis of the algal biomass.

e. Single Chain Antibodies to Facilitate Organism Consortiums

In one further embodiment of the present invention, the single chain antibodies are used to create synthetic organism consortiums on biofilms that exchange nutrients and bioproduct molecules. The use of organism consortiums facilitates processing and efficiency. In one embodiment of the present invention, the biofilm or substrate captures, on one side, a first organism, e.g., algae capable of producing and secreting metabolites such as sugars, as described above. The other side of the biofilm captures a different type of organism that synthetizes a desired product. The second organism utilizes the nutrients secreted by the first organism.

### EXAMPLES

In order to generate single chain antibody libraries, two alpaca research animals were immunized (named Desi & Sedona at the Tufts Cummings School of Veterinary Medicine) with total protein extracts from five different microalgal species: Chlamydomonas reinhardtii UTEX 2244, Chlorella variabilis NC64A, Coccomyxa sp. C-169, Nannochloropsis oceanica OZ-1, and Thalassiosira pseudonana CMMP 1335. For each of the organisms used, complete genomes exist and potential closely related or identical species are available for biofuels production. Desi received only C. reinhardtii and Sedona received the other four algae. 500 mg of algae was injected after freezing, thawing, and sonication and dilution in 1 ml of sterile normal saline.

The immunizations proceeded in four injection cycles at two-week intervals without adverse effect to the animals. Two weeks after the last injection serum was harvested from each animal for establishment of immune response by ELISA for serum antibody. The following protocol was followed for the ELISA tests. Healthy C. reinhardtii or Chlorella variabilis cells were sonicated with or without SDS and whole cell lysate was prepared in PBS buffer. 2.5 μL of the lysate was loaded into each well with 1000 μL coupling buffer (0.1 M NaHCO₃ pH 8.6+0.4 M NaCl, pH 8.6) and mixed well. The lysate was left for 2 hrs at room temperature and 5 hrs at 4° C. All the following steps were processed on a shaker in room temperature. The lysate was blocked with 1 mL PBS with 2.5% milk for 4 hrs, washed 2 times each time 1.5 mL by PBST. Dilutions of 1:2000 of each VHH (1 μM to 1 pM) were loaded on the well with PBS buffer, incubated for 4 hrs, and washed twice by PBS. The mixture was incubated with 1:2000 dilution of Goat anti-e-tag secondary antibody conjugated with HRP (Bethyl Labs) in each well with PBS buffer for 1 hr, washed 2 times with PBS; 200 μL of TMB (Sigma) was added to each well for 10 min until blue color was stable, then 200 μL 1 N HCl for 10 min to cease the colorimetric reaction. The reaction mix was placed in a centrifuge and supernatant was measured with OD at 450 nm.

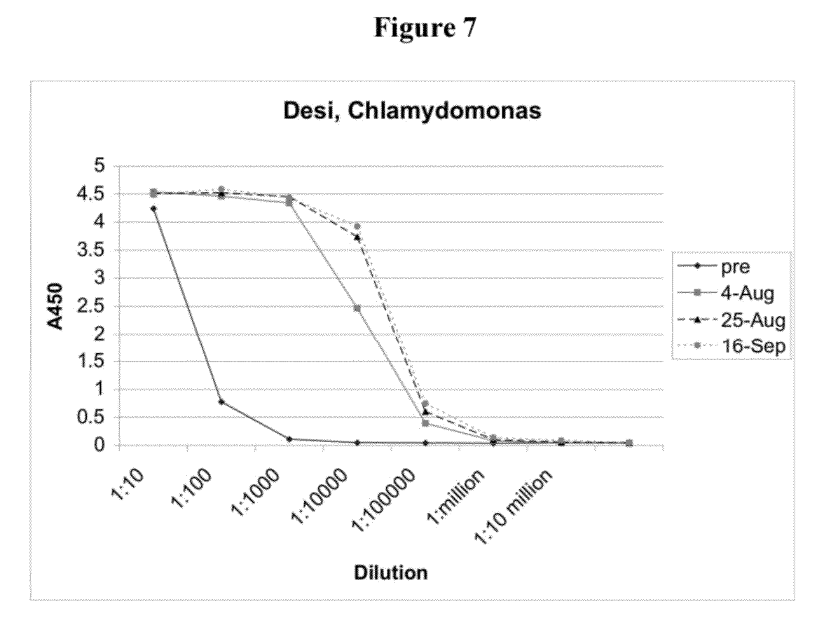

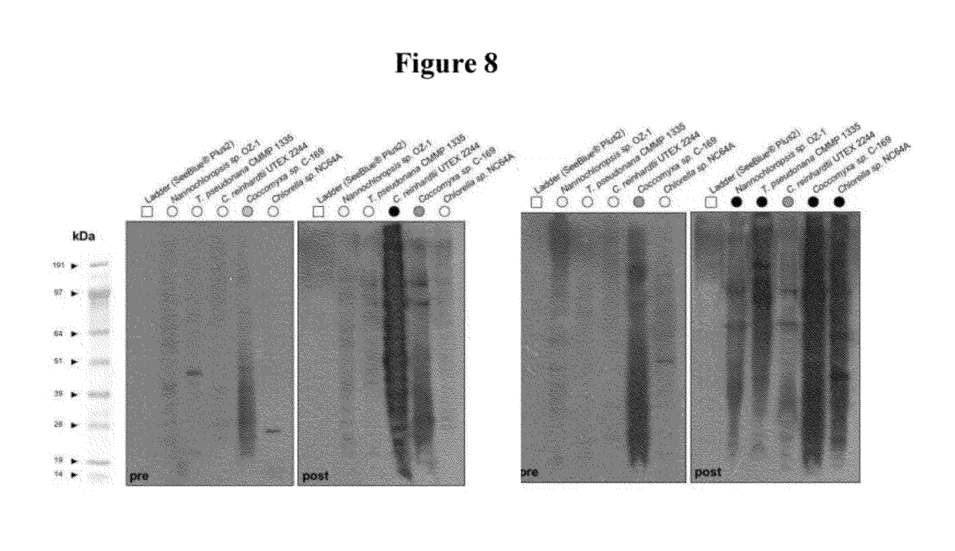

This procedure was performed with polystyrene microtiter plates with wells coated with intact algae from C. reinhardtii, Nannochloropsis oceanica, Chlorella variabilis NC64A, or Chlorella sorokiniana CCTCC M209220. Desi who was injected with C. reinhardtii only showed a strong immune response by ELISA to C. reinhardtii (FIG. 7) but not Nannochloropsis oceanica, Chlorella, Coccomyxa, and T. pseudonana (data not shown), as expected. Similarly, Sedona (injected with Nannochloropsis oceanica, Chlorella NC64A, Coccomyxa C-169, and T. pseudonana) demonstrated a good response to Chlorella, Nannochloropsis oceanica, Coccomyxa, and T. pseudonana, but not Chlamydomonas (data not shown). The sera response of these animals was also analyzed by Western blot analysis with pre-immunization as well as subsequent bleeds post-immunization and shows specific immunoreactivity with the respective algae as shown in FIG. 8.

Based on these positive results peripheral lymphocytes were harvested and phage display libraries encoding the VHH domain of the heavy chain only antibodies were generated. For the purpose of generating algae-tethering single chain antibodies, the phage library was panned against intact production algae organisms to identify a small number of species-specific and high affinity single chain antibodies to the algal surface. These single chain antibody cDNA clones were isolated and expressed as fusion proteins in E. coli. Other suitable hosts may be used to allow further characterization.

In one exemplary embodiment of the method described above, nine clones expressing small chain antibodies specific for C. reinhardtii were isolated and the sequences of the single chain antibodies isolated from those clones are provided herein in the Sequence Listing below, which is incorporated herein in its entirety. As shown in FIG. 7, the single chain antibodies disclosed above have 40% identical amino acids and maintain their function of binding to C. reinhardtii.

Once the single chain antibodies are isolated, they are expressed as fusions to the yeast Aga2 protein and displayed on the surface of yeast. It is contemplated that the proteins may also be used in fusion proteins for display in the surface of selected microorganism, such as an algae species, a diatom, a cyanobacterium and other photosynthetic microorganisms.

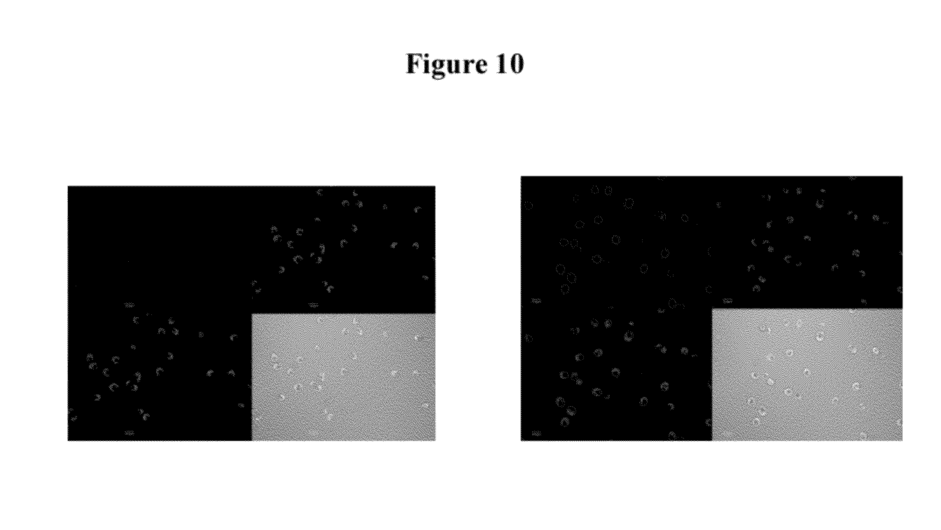

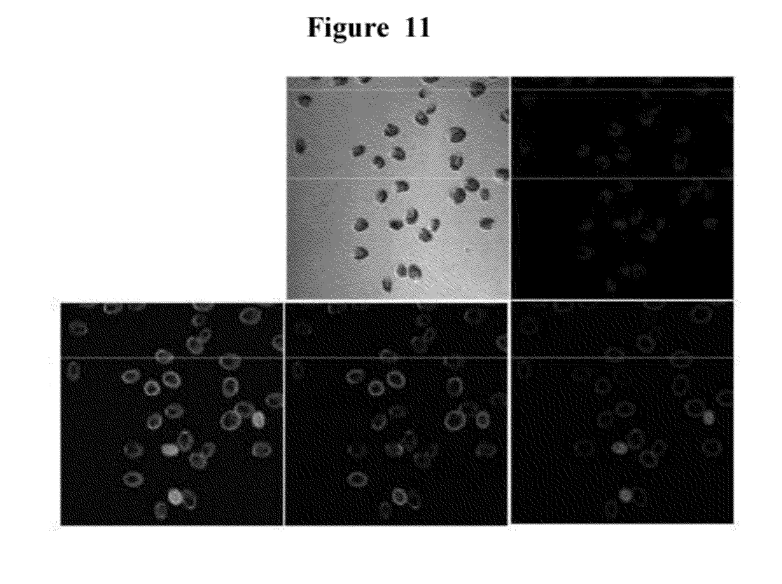

In another example of the present invention, a construct comprising a substrate binding domain, a fluorescent marker, the single chain antibody, and an epitope tag. The sequence of two exemplary constructs are provided as SEQ ID Nos. 73 and 74. The construct of SEQ ID No. 73 comprises the following components Trx-6xHis-GFP-VHH(SEQ ID No. 9)-E Epitope tag. The construct of SEQ ID No. 74 comprises the following components Trx-6xHis-mCherry-VHH(SEQ ID No. 6)-E Epitope tag. The constructs were used to show that the single chain antibodies effectively bind their algae targets. As shown on FIG. 10, the construct of SEQ ID No. 74 attaches to C. reinhardtii. The left panel shows the cells in the absence of the construct. The right panel shows the cells after addition of the construct. FIG. 11 shows confocal images of wild type Chlamydomonas reinhardtii (cc124) show GFP signal (green) and mCherry signal (red) in cell wall. The cells were treated with GFP-tagged JGJ-B11 VHH first and then with mCherry-tagged JGK-H10 after washing. The blue portion represent the chlorophyll autofluorescence from the chloroplasts of the cells. JGJ-B11 shows polarity at the flagellar end of the cell.

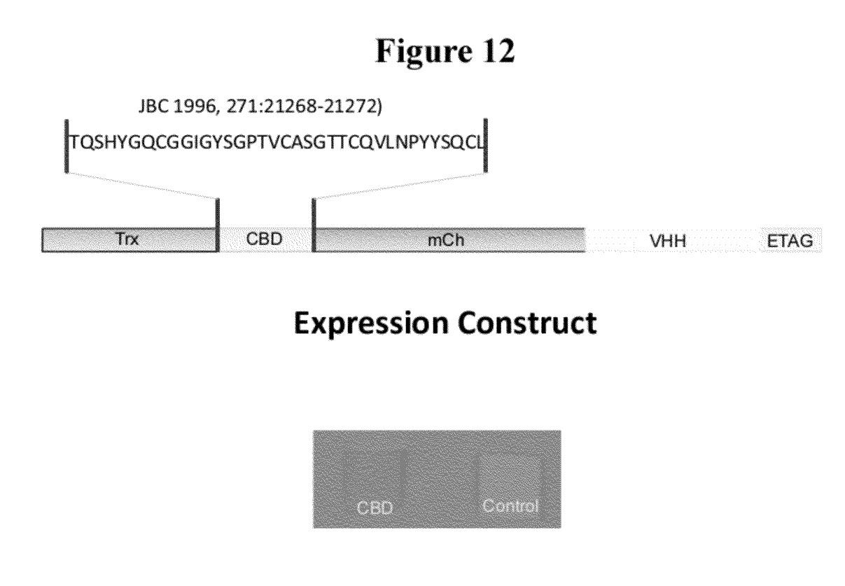

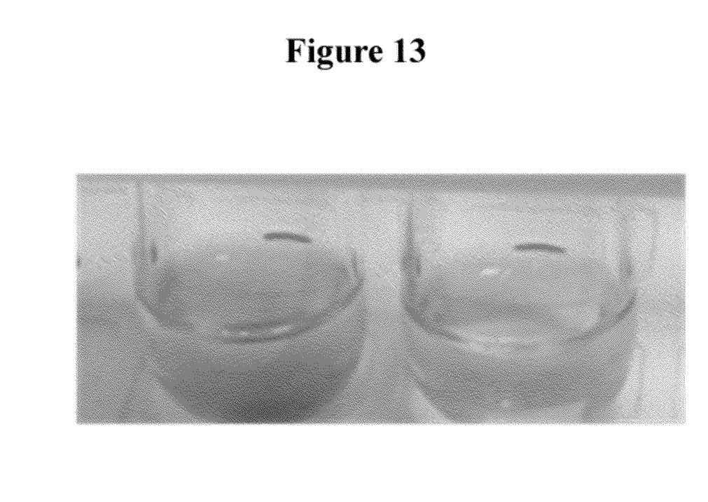

As shown in FIG. 12, a construct in accordance with one embodiment of the present invention shows binding of the construct to a cellulose membrane. The Figure shows the accession number to the substrate binding domain, i.e., the cellulose binding domain (CBD). The Figure also shows binding to the substrate. The left side substrate is pink, showing expression of the mCherry marker, while the control is white. FIG. 13 shows different cell density of Chlamydomonas reinhardtii cells in a). culture was inoculated by a piece of Whatman paper pretreated with CBD-mCherry-VHH(JGJ-B11) protein and followed by incubated with live C. reinhardtii cells and b). the same treatment but pretreated with CBD-mCherry control protein. Image was taken three days post the inoculation.

The single chain antibodies of the present invention were shown to be able to recognize different species of algae. For example, the alpaca immunized with Chlorella variabilis NC64A produced single chain antibodies capable of recognizing Chlorella sorokiniana CCTCC M209220 after panning with (CS-01). The nine Chlorella VHHs described herein were the result of panning on NC64A for two cycles of panning. On the third panning, the phage was panned and selected on NC64A for both species. 100 clones from NC64A were selected for ELISA on NC64A extract and about 75% were positive. 200 clones were selected from CS-01 panning for ELISA on CS-01 extract and about 50% were positive. 19 clones were fingerprinted as described above, moderate to strong positives from each were recovered. Although some fingerprints were enriched on one species or the other, it was clear that the same clones were commonly observed following selection on both species.

The invention has been described with references to preferred embodiments. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

### REFERENCES

The following references cited in the specification are hereby incorporated by reference in their entirety.
M J Ripin. Evaluation of immobilized cell systems for the production of fuels from microalgae. In: Aquatic Species Program Review, Proceedings of the March 1985 Principal Investigators Meeting. Golden, Colo.: SERI/CP-231-2700, Solar Energy Research Institute; 1985.
Hameed M S A and Ebrahim O H. Biotechnological Potential Uses of Immobilized Algae. International Journal or Agriculture & Biology 2007, 9:183-192.
Johnson M B and Wen Z. Development of an attached microalgal growth system for biofuel production. Appl Microbiol Biotechnol 2010, 85:525-534.
Muñoza R, Köllnera C and Guieyssea B. Biofilm photobioreactors for the treatment of industrial wastewaters. Journal of Hazardous Materials 2009, 161: 29-34.
Saerens D, Ghassabeh G H and Muyldermans S. Single-domain antibodies as building blocks for novel therapeutics. Current Opinion in Pharmacology 2008, 8:600-608.
A S Lee, M Mahapatro, D A Caron, A A G Requicha, B A Stauffer, M E Thompson, C Zhou. (2006) Whole-Cell Sensing for a Harmful Bloom-Forming Microscopic Alga by Measuring Antibody—Antigen Forces. IEEE Transactions on Nanobioscience. 5(3):149-156.
Tormo J, Lamed R, Chirino A J, Morag E, Bayer E A, Shoham Y, Steitz T A. (1996) Crystal structure of a bacterial family-III cellulose-binding domain: a general mechanism for attachment to cellulose. EMBO J. 15: 5739-5751
Yamada T, Kawasaki T, Nishida K, Hiramatsu S. (1999) Chlorella virus as a source of novel enzymes. Journal of Bioscience and Bioengineering. 88(4): p. 353-361.
Chuchird N, Sugimoto I, Fujie M, Usami S, Yamada T. (2001) Digestion of Chlorella cells by Chlorovirus-encoded polysaccharide degrading enzymes. Microbes and Environments. 16(4):206-212.
Sugimoto I, Murakami D, Fujie M, Usami S, Yamada T. (2000) Algal-lytic activities encoded by Chlorella virus CVK2. Virology. 277:119-126.
Hiramatsu S, Fujie M, Usami S, Yamada T. (1999) Expression of a chitinase gene and lysis of the host cell wall during Chlorella virus CVK2 infection. Virology. 260:308-315.
Maass, D. R., Sepulveda, J., Pernthaner, A., Shoemaker, C. B., 2007. Alpaca (Lama pacos) as a convenient source of recombinant camelid heavy chain antibodies (VHHs). J. Immunol. Methods 324, 13-25.

Arbabi-Ghahroudi M, Tanhn J, MacKenzie R. Prokaryotic expression of antibodies. Cancer and Metastasis Reviews 2005. 24: 501-519.
Muyldermans S. Single domain camel antibodies: current status. Reviews in Molecular Biotechnology 2001. 74:277-302.
Liva X, Brune D, Vermaas W, Curtiss R. (2010) Production and secretion of fatty acids in genetically engineered cyanobacteria. PNAS Ahead of Print.
Reppas N B, Ridley C P. (2010) Methods and compositions for the recombinant biosynthesis of n-alkanes, U.S. Pat. No. 7,794,969
Boder E T, Wittrup K D. (1997) Yeast surface display for screening combinatorial polypeptide libraries. Nature Biotechnology 15:553-557.
Lamed R, Setiter E, Bayer E A. (1983) Characterization of a Cellulose-Binding, Cellulase-Containing Complex in Clostridium thermocellum. Journal of Bacteriology 15:828-836.
Kakiuchi M, Isui A, Suzuki K, Fujino T, Fujino E, Kimura T, Karita S, Sakka K, Ohmiya K. (1998) Cloning and DNA Sequencing of the Genes Encoding Clostridium josui Scaffolding Protein CipA and Cellulase CelD and Identification of Their Gene Products as Major Components of the Cellulosome. Journal of Bacteriology 180:4303-4308.
Fierobe H-P Mechaly A, Tardif C, Belaich A, Lamed R, Shoham Y, Belaich J-P, Bayer E A.

(2001) Design and Production of Active Cellulosome Chimeras. Journal of Biological Chemistry 275(24):2125721261.
Shoham Y, Lamed R, Bayer E A. (1999) The cellulosome concept as an efficient microbial strategy for the degradation of insoluble polysaccharides. Trends in Microbiology 7:275-281
Rosenberg J N, Oyler G A, Wilkinson L, Betenbaugh M J. (2008) A green light for engineered algae: redirecting metabolism to fuel a biotechnology revolution. Curr Opin Biotechnol 19:430-436.
Bentley H. K and Melis A. (2011) Diffusion-based process for carbon dioxide uptake and isoprene emission in gaseous/aqueous two-phase photobioreactors by photosynthetic microorganisms. Biotechnology & Bioengineering: 109: 100-109.
Lindberg P., Park S., Melis A. (2010) Engineering a platform for photosynthetic isoprene production in cyanobacteria, using Synechocystis as the model organism. Metabolic Engineering. 12: 70-79.

1. An isolated single chain antibody capable of binding an alga cell.

2. The isolated single chain antibody of claim 1, wherein the alga cell is selected from the group consisting of Chlamydomonas reinhardtii, Chlorella variabilis, Coccomyxa, Nannochloropsis oceanica, and Thalassiosira pseudonana.

3. The isolated single chain antibody of claim 1, comprising an amino acid sequence selected from the group consisting of Sequence ID. Nos. 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

4. The isolated single chain antibody of claim 3, wherein the amino acid sequence is encoded by a nucleic acid selected from the group consisting of Sequence ID Nos. 10, 11, 12, 13, 14, 15, 16, 17, 18, 28, 29, 30, 31, 32, 33, 34, 35, 36, 37, 52, 53, 54, 55, 56, 57, 58, 59, 60, 61, 62, 63, 64, 65, 66, 70, 71, and 72.

5. The isolated single chain antibody of claim 1, wherein said single chain antibody has an 85% amino acid sequence identity to a sequence selected from the group consisting of Sequence ID Nos. 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

6. The isolated single chain antibody of claim 1, wherein said single chain antibody has an 40% amino acid sequence identity to a sequence selected from the group consisting of Sequence ID Nos. 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

7. A method for isolating single chain antibodies specific for algae, the method comprising the steps of:
immunizing an animal in the camelid family with an algae strain,
collecting blood sample from the animal;
preparing a cDNA library from the blood sample;
expressing the cDNA library on a phage;
panning the phage for antibodies specific to live algae strains; and
isolating the antibodies identified in the panning step.

8. The method of claim 7, wherein the panning step comprises the following steps:
obtaining a pellet of live algae;
adding blocking reagents to the pellet;
exposing the phage to the pellet; and
eluting the phage.

9. The method of claim 7, wherein the isolating step consists of:
selecting single chain antibodies with a specific restriction endonuclease finger print, and
a positive signal from an ELISA.

10. A chimeric fusion peptide construct, comprising:
an isolated single chain antibody domain capable of binding an alga cell, and
a substrate binding domain.

11. The chimeric fusion peptide construct of claim 10, wherein the alga cell is selected from the group consisting of Chlamydomonas reinhardtii, Chlorella variabilis, Coccomyxa sp, Nannochloropsis oceanica, and Thalassiosira pseudonana.

12. The chimeric fusion peptide construct of claim 10, wherein the isolated single chain antibody domain has an amino acid sequence selected from the group consisting of Sequence ID. Nos: 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

13. The chimeric fusion peptide construct of claim 12, wherein the amino acid sequence is encoded by a nucleic acid selected from the group consisting of Sequence ID Nos. 10, 11, 12, 13, 14, 15, 16, 17, 18, 28, 29, 30, 31, 32, 33, 34, 35, 36, 37, 52, 53, 54, 55, 56, 57, 58, 59, 60, 61, 62, 63, 64, 65, 66, 70, 71, and 72.

14. The chimeric fusion peptide construct of claim 11, wherein said single chain antibody has an 85% amino acid sequence identity to a sequence selected from the group consisting of Sequence ID Nos. 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

15. The chimeric fusion peptide construct of claim 11, wherein said single chain antibody has an 40% amino acid sequence identity to a sequence selected from the group consisting of Sequence ID Nos. 1, 2, 3, 4, 5, 6, 7, 8, 9, 19, 20, 21, 22, 23, 24, 25, 26, 27, 37, 38, 39, 40, 41, 42, 43, 44, 45, 46, 47, 48, 49, 50, 51, 67, 68, and 69.

16. The chimeric fusion peptide construct of claim 1, wherein the substrate binding domain is selected from the group consisting of a cellulose binding domain, a glutenin binding domain, lectin binding domain, and fibronectin binding domain.

17. The chimeric fusion peptide construct of claim 1, further comprising a linker between the single chain antibody domain and the substrate binding domain.

18. The chimeric fusion peptide construct of claim 15, wherein the linker has a length of 5 to 50 amino acids.

19. The chimeric fusion peptide construct of claim 15, wherein the linker has a length of 10 to 40 amino acids, more preferably 20 to 30 amino acids.

20. The chimeric fusion peptide construct of claim 15, wherein the linker comprises amino acids 1 to 206 of human SNAP25a, with the cysteine residues mutated to serine, or amino acids 140 to 206 of SNAP25.

21-56. (canceled)


SEQUENCE LISTING (74 sequences)

SEQ ID NO: 1 - protein, 134 aa, Lama pacos
  PEPTIDE (9)..(125): Clone JGI-E8 Binds Chlamydomonas reinhardtii UTEX 2244
AAQVQLVESGGGMVQAGASLRLSCAASGRTFTIYDMGWFRQAPGKEREFVGIIGRRGITTDYADSVKGRFTISRDNAKKTVYLQMNSLKPEDTAVYYCAVGGSPGRLRVGVPDYWGQGTLVTVSSEPKTPKPQP

SEQ ID NO: 2 - protein, 126 aa, Lama pacos
  PEPTIDE (9)..(117): Clone JGJ-B1 Binds Chlamydomonas reinhardtii UTEX 2244
AAQLQLVETGGGLAQPGGSLSLSCELSGGTFSRGTMGWYRQAPGKQRELVASMISGDYINIVDSVKGRFTISRDNAKNTVYLQMNSLKPEDTAVYFCKYNFEGLAYWGQGTQVTVSSEPKTPKPQP

SEQ ID NO: 3 - protein, 137 aa, Lama pacos
  PEPTIDE (9)..(128): Clone JGJ-B10 Binds Chlamydomonas reinhardtii UTEX 2244
AAQVQLVETGGGLVQPGGSLRLSCAASEFTLNYRPIGWFRQAPGKEREGVSCINSSGDSTNYADSVKGRFTISRDNAKNTVYLQMNSLKPEDTAVYFCAADRSLFGVCGLSRSQYDFWGQGTQVTVSSEPKTPKPQP

SEQ ID NO: 4 - protein, 136 aa, Lama pacos
  PEPTIDE (9)..(127): Clone JGI-FI Binds Chlamydomonas reinhardtii UTEX 2244
AAQVQLVETGGGLVQPGGSLRLSCAASGFTFSTYWMYWVRQAPGKGLEWVSAIGTLGNTHYADSVKGRFTISRDNAKNTLYLQMNSLKPEDTALYYCARDLDGSSWYLKPPAVLDSRGQGTQVTVSSEPKTPKPQP

SEQ ID NO: 5 - protein, 127 aa, Lama pacos
  PEPTIDE (9)..(118): Clone JGJ-C3 Binds Chlamydomonas reinhardtii UTEX 2244
AAQLQLVETGGGLVQAGGSLRLSCAASGNIFRVRAMAWYRQAPGKQREWVAVLGSRGGTNYADSVKGRFTISRDNAANTIFLQMNSLKPEDTAVYYCNHYPPEKRDYWGQGTQVTVSSEPKTPKPQP

SEQ ID NO: 6 - protein, 127 aa, Lama pacos; identical to SEQ ID NO: 5
  PEPTIDE (9)..(118): Clone JGK-H10 Binds Chlamydomonas reinhardtii UTEX 2244

SEQ ID NO: 7 - protein, 128 aa, Lama pacos
  PEPTIDE (9)..(119): Clone JGI-F5 Binds Chlamydomonas reinhardtii UTEX 2244
AAQVQLVETGGGLVQAGESLRLSCVASGFTFSRHPMAWYRQAPGKARELVASIASSSGVTDYHASVRGRFTISRDNAKNTVYLQINILKPEDTAAYYCNALPNLPRNYWGQGTQVTVSSEPKTPKPQP

SEQ ID NO: 8 - protein, 137 aa, Lama pacos; identical to SEQ ID NO: 3
  PEPTIDE (9)..(128): Clone JGJ-B12 Binds Chlamydomonas reinhardtii UTEX 2244

SEQ ID NO: 9 - protein, 136 aa, Lama pacos
  PEPTIDE (9)..(127): Clone JGJ-B11 Binds Chlamydomonas reinhardtii UTEX 2244
AAQVQLVETGGGLVQAGGSLRLSCVVSGRNNSGIGMGWFRQAPGKEREFVAAIGWGGSSTIYADSVKGRFTVSRDNAKNTVYLQMVSLKPDDTAVYVCAARRRALTPISLAAGFEYWGQGTQVIVSAEPKTPKPQP

SEQ ID NO: 10 - DNA, 404 bp, Lama pacos
  misc_feature: Clone JGI-E8
ggccgctcaggtgcagctcgtggagtcgggaggaggaatggtgcaggctggggcctctctgagactctcctgtgcagcctctggacgcaccttcactatctatgacatgggctggttccgccaggctccagggaaggagcgtgagtttgtaggaattataggtcggagaggtattaccacagactatgcggactccgtgaagggccgattcaccatctccagagacaacgccaagaagacggtgtatctacaaatgaacagcctgaaacctgaggacacggccgtttattactgcgcagtaggtggaagtccggggcgactacgggttggggtacctgactactggggccaggggaccctggtcaccgtctcctcagaacccaagacaccaaaaccacaaccgg

SEQ ID NO: 11 - DNA, 380 bp, Lama pacos
  misc_feature: Clone JGJ-B1
ggccgctcagttgcagctcgtggagacgggcggaggcttggcgcagcctggggggtctctgagcctctcctgcgaactttctggagggaccttcagtaggggtaccatgggctggtaccgccaggctccagggaagcagcgcgagttggtcgcatctatgattagtggtgactacataaacattgtagactccgtgaagggccgattcaccatctccagagacaacgccaagaacacggtgtatctgcaaatgaacagcctgaaacctgaggacacggccgtctatttttgtaaatacaacttcgagggacttgcctactggggccagggaacccaggtcaccgtctcctcagaacccaagacaccaaaaccacaaccgg

SEQ ID NO: 12 - DNA, 413 bp, Lama pacos
  misc_feature: Clone JGJ-B10
ggccgctcaggtgcagctcgtggagacggggggaggcttggtgcagcctggggggtctctgagactctcctgtgcagcctctgaattcactttgaattatagacccataggctggttccgccaggccccagggaaggagcgtgagggggtctcatgtattaatagtagtggtgatagcacaaactacgcggactccgtgaagggccgattcaccatctccagagacaacgccaagaacacggtgtatctgcaaatgaacagcctgaaacctgaggacacagccgtttatttctgtgcagcagatcggagtctgttcggagtatgtgggttatctcgttcccagtatgatttctggggccaggggacccaggtcactgtttcctcagaacccaagacaccaaaaccacaaccgg

SEQ ID NO: 13 - DNA, 410 bp, Lama pacos
  misc_feature: Clone JGI-F1
ggccgctcaggtgcagctcgtggagacagggggaggcttggtgcagcctggggggtctctgagactctcctgtgcagcctctggattcaccttcagtacctactggatgtattgggtccgtcaggctccagggaaggggctcgagtgggtctcagctattggtacgttgggtaacacacactatgcagactccgtgaagggccgattcaccatctccagagacaacgccaagaacacgctgtatctgcaaatgaacagcctgaaacctgaggacacggccctgtattactgcgcgagagacttggacggtagtagctggtatttgaaaccccctgcggtgcttgactcgcggggccaggggacccaggtcaccgtctcctcagaacccaagacaccaaaaccacaaccgg

SEQ ID NO: 14 - DNA, 383 bp, Lama pacos
  misc_feature: Clone JGJ-C3
ggccgctcaggtgcagctcgtggagtctgggggaggcctggtgcaggctggggggtctctgagactctcctgtgtagcctctggaagcgccttcagtaaagatgtctgggcctggtaccgccaggctccagggaaacagcgcacgtgggtcgccgtaattggtagtgccggcggcaccaactatgcagagtccgtgaagggccgattcaccatctccagagagaacgccaagaacacggtgtatctacaaatgaacagtctaaagccagaagacacagccaaatattattgtaataaatttcccgacgtaagaggccgtaactggggccaggggacccaggtcaccgtctcctcggaacccaagacaccaaaaccacaaccgg

SEQ ID NO: 15 - DNA, 383 bp, Lama pacos
  misc_feature: Clone JGK-H10
ggccgctcagttgcagctcgtggagactgggggaggcttggtgcaggctggggggtctctgagactctcctgtgcagcctctggcaacatcttccgtgtccgtgccatggcttggtaccgccaggctccagggaagcagcgcgagtgggtcgcagttcttggtagtagaggtggtacaaactatgcagactccgtgaagggccgtttcaccatctccagagacaacgccgcgaacacgatatttctgcaaatgaacagcctgaaacctgaggacacagccgtctattactgtaatcactaccctccggaaaaacgggactactggggccaggggacccaggtcaccgtctcctcagaacccaagacaccaaaaccacaaccgg

SEQ ID NO: 16 - DNA, 386 bp, Lama pacos
  misc_feature: Clone JGI-F15
ggccgctcaggtgcagctcgtggagacggggggaggcttggtgcaggctggggagtctctgagactctcctgtgtagcctctggatttaccttcagtcgccatcccatggcctggtaccgccaggctccaggaaaggcgcgcgagctggtcgcgtcgattgctagtagtagtggtgtgactgactatcacgcttccgtgaggggccgattcaccatctccagagacaacgccaagaacacggtgtatctacaaataaacatcctgaaacctgaggacacagccgcctactactgtaatgcactcccgaatttaccaaggaactactggggccagggaacccaggtcaccgtctcctcagaacccaagacaccaaaaccacaaccgg

SEQ ID NO: 17 - DNA, 413 bp, Lama pacos; identical to SEQ ID NO: 12
  misc_feature: Clone JGJ-B12

SEQ ID NO: 18 - DNA, 410 bp, Lama pacos
  misc_feature: JGJ-B11
ggccgctcaggtgcagctcgtggagacggggggaggattggtgcaggctgggggctctctgagactctcctgtgtagtctctggacgcaacaatagtggcattggcatgggctggttccgccaggctccgggtaaggaacgtgaatttgtagcagctattggttggggcggtagtagtacgatctatgcagattccgtgaagggccgattcaccgtctccagagacaacgccaagaacacggtgtatctacaaatggtcagcctgaaacctgacgacacggccgtttatgtctgcgcagcgagaagacgcgcgctcactccaatttctctcgccgccgggtttgagtactggggccaggggacccaggtcatcgtctccgcagaacccaagacaccaaaaccacaaccgg

SEQ ID NO: 19 - protein, 138 aa, Lama pacos
  misc_feature (9)..(130): Clone JGN-D5 Binds Chlorella variabilis NC64A
AAQVQLVETGGGLVQPGGSLRLSCAVSGLDLGEHAIGWFRQAPGKEREGVLCISNRGNTADFPTSPVSKGGRFAISRDVAKSVVYLQINDLKLEDTANYSCAATYSFYYCPTHWTDDVYWGQGTQVTVASAHHSEDPS

SEQ ID NO: 20 - protein, 132 aa, Lama pacos
  PEPTIDE (9)..(123): Clone JGP-C4 Binds Chlorella variabilis NC64A
AAQLQLVESGGGLVQAGGSLRLSCAASGNIFSINVMGWYRQAAGEQRELVAVITDGGYSEYSDSVKGRFTISRDIAKNTVYLQMNSLKPEDTAAYYCYARAVSTRSQWYSEVWGQGTLVTVSSEPKTPKPQP

SEQ ID NO: 21 - protein, 127 aa, Lama pacos
  PEPTIDE (9)..(118): Clone JGP-H4 Binds Chlorella variabilis NC64A
AAQVQLVESGGGLVQAGGSLRLSCAASGSIFSINAMGWYRQPPGKQRELVARIINGSDSPMYADSAKGRFTISKDNAKNTVYLQMNSLKPEDTAVYSCWAVVNDMGYWGKGTLVTVSSEPKTPKPQP

SEQ ID NO: 22 - protein, 144 aa, Lama pacos
  PEPTIDE (9)..(123): Clone JGP-C1 Binds Chlorella variabilis NC64A
AAQLQLVESGGGLVQSGGSMSLSCTARSSFSVMGYYRQAPGKERELVALIARNRDTKYAESVKGRFTISRDNTKNTVYLQMNNLGPEDTAVYYCNAGTSRLSISTGTPGTTWGPGTQVTVSSSESSRPGTQVTASSAHHSEDPS

SEQ ID NO: 23 - protein, 130 aa, Lama pacos
  PEPTIDE (9)..(121): Clone JGN-F5 Binds Chlorella variabilis NC64A
AAQVQLVESGGGSVQAGGSLTLSCEVPAAMMSENIMGWFRQAPGKSRVFVANIVSGSDKVHIADAVQGRFTISRDNAKNTVYLHMSQLKLEDTAVYYCNLKAWTPTYSETWGQGMQVTVSSEPKTPKPQP

SEQ ID NO: 24 - protein, 127 aa, Lama pacos
  PEPTIDE (9)..(118): Clone JGN-B9 Binds Chlorella variabilis NC64A
AAQLQLVESGGGLVQAGGSLRLACSASGSIGPFAPMGWYRQAPGKQRELVAGISSDGTTTYADSVKGRFTISRDNNDKITVYLQMNSLRPEDTAVYHCNGYENWSAPWGQGTQVTVSTEPKTPKPQP

SEQ ID NO: 25 - protein, 128 aa, Lama pacos
  PEPTIDE (9)..(119): Clone JGP-C6 Binds Chlorella variabilis NC64A
AAQVQLVESGGGLVQTGESLRLSCVASGFTFSRHPMAWYRQAPGKARELVASIASSSGMTDYHDSVKGRFTISRDNAKNTVYLQMNSLKPEDTAAYYCNALPTLPRNYWGQGTQVTVSSEPKTPKPQP

SEQ ID NO: 26 - protein, 127 aa, Lama pacos
  PEPTIDE (9)..(110): Clone SEQ ID No. 26 Binds Chlorella variabilis NC64A
AAQVQLVESGGGLVQAGGSLRLSCVASGSAFSADVMAWYRQAPGKQRTWVAVIGSAGGTNYADSVKGRFTISRENAKNTVYLQMNSLKAEDTAVYYCNKFPDLRGRNWGQGTQVTVSSEPKTPKPQP

SEQ ID NO: 27 - protein, 135 aa, Lama pacos
  PEPTIDE (9)..(126): Clone JGN-A3 Binds Chlorella variabilis NC64A
AAQVQLVETGGGLVQAGASLRLSCVDSGGIFWSSIMAWFRQAPGQEREFVSAITWTGDSTYYEDSVKGRFTISRDNAKNTVYLQMNGLKPGDTAVYYCAASPSTVVGRAAREYPYWGQGTQVTVSSEPKTPKPQP

SEQ ID NO: 28 - DNA, 416 bp, Lama pacos
  misc_feature: Clone JGN-D5
ggccgctcaggtgcagctcgtggagacggggggaggcttggtgcagcctggggggtctctgagactctcctgtgcggtctctggattggatttgggggagcatgccataggctggttccgccaggccccaggaaaggagcgtgagggagtcctatgtattagcaatcgtgggaatacggctgactttccgacttctcctgtttccaaggggggtcgattcgccatctccagagacgtcgccaagagtgtggtctatctacaaatcaacgacttgaaactggaggacacagccaactattcttgtgcagcaacctatagtttttactattgtcccacgcactggaccgacgatgtgtattggggccagggaacccaggtcaccgtcgcctcggcgcaccacagcgaagacccctcgg

SEQ ID NO: 29 - DNA, 398 bp, Lama pacos
  misc_feature: Clone JGP-C4
ggccgctcagttgcagctcgtggagtcggggggaggcttggtgcaggctggggggtctctgagactctcctgtgcagcctctggaaacatcttcagtatcaatgtcatgggctggtaccgccaggctgcaggggagcagcgcgagttggtcgcagtcattactgacggtggttacagcgagtattcagactccgtgaagggccgattcaccatctccagagacatcgccaagaacacggtgtatctacaaatgaacagcctgaaacctgaggacacagccgcctattactgctatgcgagagccgtcagtaccaggagtcaatggtacagcgaagtttggggccagggcaccctggtcactgtctcctcagaacccaagacaccaaaaccacaaccgg

SEQ ID NO: 30 - DNA, 383 bp, Lama pacos
  misc_feature: Clone JGP-H4
ggccgctcaggtgcagctcgtggagtcggggggaggcttggtgcaggctggggggtctctgagactctcctgtgcagcctctggaagcatctttagtatcaatgccatgggctggtaccgccagcctccagggaaacagcgcgagttggtcgctcgtattattaacggtagcgacagtccaatgtatgcagattccgcgaagggccgattcaccatctccaaagacaacgccaagaacacggtgtatctgcaaatgaacagcctgaaacctgaggacacagccgtctattcttgttgggcggtagttaatgatatgggctactggggcaaagggaccctggtcaccgtctcctcagaacccaagacaccaaaaccacaaccgg

SEQ ID NO: 31 - DNA, 434 bp, Lama pacos
  misc_feature: Clone JGP-C1
ggccgctcagttgcagctcgtggagtctggtggaggcttggtgcagtctggggggtcaatgtcactctcctgcacagcccgttcaagcttcagtgttatgggatactaccgccaggctccagggaaggagcgcgagttggtcgcgcttattgcccgtaatagagatacaaagtatgcggagtccgtgaagggtcgattcaccatctccagagacaacaccaagaatacggtgtatttgcaaatgaacaacctgggccctgaggacacggccgtctattactgtaatgcaggaacatcacgactatcgatttccactggtacgccggggacgacctggggcccggggacccaggtcaccgtctcctcgagtgagtcctctcgcccggggacccaggtcaccgcctcctcggcgcaccacagcgaagacccctcgg

SEQ ID NO: 32 - DNA, 392 bp, Lama pacos
  misc_feature: Clone  JGN-F5
ggccgctcaggtgcagctcgtggagtcggggggaggctcggtacaggctggggggtctcttaccctctcctgtgaagtccctgcagcaatgatgagtgagaatatcatgggctggttccgccaggctccagggaagtcgcgcgtgttcgtggcaaatatagttagtgggagtgataaggtgcatattgcagacgccgtgcagggccggttcaccatctccagagacaacgccaagaacacggtgtatctgcacatgagccaattgaaacttgaggacacagccgtatattattgtaatctgaaggcttggacccccacttacagcgagacgtggggccagggaatgcaggtcaccgtctcctcagaacccaagacaccaaaaccacaaccgg

SEQ ID NO: 33 - DNA, 383 bp, Lama pacos
  misc_feature: Clone JGN-B9
ggccgctcagttgcagctcgtggagtccggaggaggtttagtgcaggcgggggggtctctgagactcgcctgtagtgcttctgggagtatcgggcctttcgctcccatgggctggtaccgccaggctccaggaaagcagcgcgaattggtcgcgggtattagtagtgatgggacgacaacgtatgcagactccgtgaagggccgattcaccatctccagagacaacaacgacaagattacggtgtatctgcaaatgaacagcctgagacctgaggacacagcggtctatcactgtaatggttatgagaattggagtgccccctggggccaggggacccaggtcaccgtctccacagaacccaagacaccaaaaccacaaccgg

SEQ ID NO: 34 - DNA, 386 bp, Lama pacos
  misc_feature: Clone JGP-C6
ggccgctcaggtgcagctcgtggagtcggggggaggcttggtgcagactggggagtctctgagactctcctgtgtagcctctggatttaccttcagtcgccatcccatggcctggtaccgccaggctccaggaaaggcgcgcgagctggtcgcgtcgattgctagtagtagtggtatgactgactatcacgactccgtgaagggccgattcaccatctccagagacaacgccaagaacacggtgtatctgcaaatgaacagcctgaaacctgaggacacagccgcctactactgtaatgcactcccgactctaccaaggaactactggggccagggaacccaggtcaccgtctcctcagaacccaagacaccaaaaccacaaccgg

SEQ ID NO: 35 - DNA, 383 bp, Lama pacos
  misc_feature: Clone JGN-B7
ggccgctcaggtgcagctcgtggagtcgggcggaggcctggtgcaggctggggggtctctgagactctcctgtgtagcctctggaagcgccttcagtgcagatgtcatggcctggtaccgccaggctccagggaaacagcgcacgtgggtcgccgtaattggtagtgccggcggcacaaactatgcagactccgtgaagggccgattcaccatctccagagagaacgccaagaacacggtgtatctacaaatgaacagtctgaaagcagaagacacagccgtctattattgtaataaattccccgacctaagaggccgtaactggggccaggggacccaggtcaccgtctcctcggaacccaagacaccaaaaccacaaccgg

SEQ ID NO: 36 - DNA, 407 bp, Lama pacos
  misc_feature: Clone JGN-A3
ggccgctcaggtgcagctcgtggagactgggggaggattggtgcaggccggggcctctctgagactctcctgtgtagactctggaggcatcttctggagctctatcatggcctggttccgccaggctccagggcaggagcgtgagtttgtatcagctattacgtggactggtgatagcacatactatgaagactccgtaaagggccgattcaccatctccagagacaacgccaagaacacggtgtatctgcaaatgaacggcctgaaacctggcgacacggccgtttactactgtgcagcaagccccagtacagtggtaggacgtgctgcccgtgaatacccatactggggccaggggacccaggtcaccgtctcctcagaacccaagacaccaaaaccacaaccgg

SEQ ID NO: 37 - protein, 137 aa, Lama pacos
  PEPTIDE (9)..(129): Clone JGQ-F7 Binds Nannochloropsis sp. OZ-1
AAQVQLVESGGGSVQPGGSLRLSCAASGFTFSSYALGWARQVPGKGLQWVSGVYSNGNTYYADSVKGRFTISRDNAKNTVYLQMDSLKPEDTAVYHCAVGGRGACTYSDGSLHCPNEYWGQGKQVTVSSAHHSEDPS

SEQ ID NO: 38 - protein, 122 aa, Lama pacos
  PEPTIDE (9)..(113): Clone JGQ-A7 Binds Nannochloropsis sp. OZ-1
AAQVQLVESGGSLRLSCAASRSLFSGNTMGWYRQAPGNEREVVARITPDGRRNYADSVKGRFTISRDNAKNTMYLEMNNLKVDDTATYYCNSVAAVLRRTASWGQGTQVTVSSEPKTPKPQP

SEQ ID NO: 39 - protein, 128 aa, Lama pacos
  PEPTIDE (9)..(119): Clone JGQ-D9 Binds Nannochloropsis sp. OZ-1
AAQLQLVETGGGLAQPGGSLRLSCAASGSISRPYAMGWYRQIPGKQREWVAHINRAGSRTNYTDSVKGRFTISRDNTKNTVYLEMNSLKPEDTAVYYCNAVPRYGRDYWGQGTQVTVSSEPKTPKPQP

SEQ ID NO: 40 - protein, 129 aa, Lama pacos
  PEPTIDE (9)..(120): Clone JGR-H5 Binds Nannochloropsis sp. OZ-1
AAQLQLVETGGGLVQTGGSLRLSCAANRSIFGISAMEWYRQAPGKDRELVARITSGGSTNYDDSVKGRFTISRDNAKNTVYLQMNSLKPEDTGVYFCNSRNGRNWSRGSWGQGTQVTVSSEPKTPKPQP

SEQ ID NO: 41 - protein, 123 aa, Lama pacos
  PEPTIDE (9)..(114): Clone JGQ-H9 Binds Nannochloropsis sp. OZ-1
AAQVQLVETGGGSVQAGGSLRLSCVASQTIYTIMAWYRQAPGKERELVASVLRDGRTNHAASVKGRFTISRDNAKNTIYLQMNSLKPEDTAIYYCNVGGFTSSWGQGTRVTVSSEPKTPKPQP

SEQ ID NO: 42 - protein, 140 aa, Lama pacos
  PEPTIDE (9)..(131): Clone JGR-G5 Binds Nannochloropsis sp. OZ-1
AAQVQLVETGGGLVQAGGSLRLSCAASGTIFNGNRINAMGWYRQIPGKERDLVATITVDGKVNLGNLVKGRFTISEDDARNTVYLQMNRLKPEDTAVYYCTAGKLTSSGGVDYYSPSNDYWGQGTQVTVSSEPKTPKPQP

SEQ ID NO: 43 - protein, 125 aa, Lama pacos
  PEPTIDE (9)..(116): Clone JGR-H9 Binds Nannochloropsis sp. OZ-1
AAQLQLVESGGGLVPAGGSLRLSCAASGSIFNGNVMAWYRRTPGNQRNMVAAITSSGSTTYPHSVKGRFTISRDNAENTLYLQMNSLKPEDTAVYYCNTLQPLNYWGQGTQVTVSSEPKTPKPQP

SEQ ID NO: 44 - protein, 126 aa, Lama pacos
  PEPTIDE (9)..(117): Clone JGR-G2 Binds Nannochloropsis sp. OZ-1
AAQLQLVETGGGLVQAGGSLRLSCLASGSIVNSQTVAWYRQAPGKSREFVAHITSQGLAGYASSVRGRFTISRNTGKNTAYLQMNSLQPEDTAVYYCTADVRGYRYWGQGTQVTVSSEPKTPKPQP

SEQ ID NO: 45 - protein, 132 aa, Lama pacos
  PEPTIDE (9)..(123): Clone JGR-G11 Binds Nannochloropsis sp. OZ-1
AAQVQLVETGGGLVQAGGSLRLSCAASGSISEIYRMGWYRQAPGKQRELVAAITSPGNTNYPDSLKGRFAISRDYAKNTGYLQMDRLEPEDTAVYYCKANLLQRAPRKYLEIWGQGTLVTVSSEPKTPKPQP

SEQ ID NO: 46 - protein, 129 aa, Lama pacos
  PEPTIDE (9)..(120): Clone JGQ-C12 Binds Nannochloropsis sp. OZ-1
AAQVQLVESGGGLVRAGGSLKLTCAVSGDIFSIKAMGWYRRAPGKKRESVAAISAAGNTLYADSVKGRFTISRDNDANTLFLQMNSLKPEDTAMYYCAAEINNSDSLNQGGQGTQVTVSSEPKTPKPQP

SEQ ID NO: 47 - protein, 135 aa, Lama pacos
  PEPTIDE (9)..(127): Clone JGR-H6 Binds Nannochloropsis sp. OZ-1
AAQVQLVESGGGLVQAGGSLRLSCAGSGRSFSAAVMGWFRQAPGKEREFVAALRQIIGSTHYADSVKGRFTISRDNAKNMLYLDMNSLKPTDTAAYYCTAGPPTMLDVSTDREYDTWGQGTQVAVSSAHHSEDPS

SEQ ID NO: 48 - protein, 134 aa, Lama pacos
  PEPTIDE (9)..(125): Clone JGR-H8 Binds Nannochloropsis sp. OZ-1
AAQVQLVETGGLVQTGGSLRLSCSASGRTFSHYTMGWFRQAPGKEREFVATISRSGGKTAYADSVKGRFTISRDNAKNMVFLQMTSLKSEDTAVYYCAASRLTGPQPLHDKYAYWGQGTQVTVSSEPKTPKPQP

SEQ ID NO: 49 - protein, 127 aa, Lama pacos
  PEPTIDE (9)..(118): Clone JGQ-B3 Binds Nannochloropsis sp. OZ-1
AAQVQLVESGGGLVQTGGSLRLSCVGSGISLTKDMMAWYRQAPGKQRELVATIPVVGGGRPTYEDSVKGRFTISRDNAKNTVYLEMNSLKPEDTGDYYCQRPSAWLAWGQGTQVTVSSEPKTPKPQP

SEQ ID NO: 50 - protein, 133 aa, Lama pacos
  PEPTIDE (9)..(125): Clone JGR-E5 Binds Nannochloropsis sp. OZ-1
AAQLQLVETGGGTVQAGGSLRLSCEASGFRFADYAIGWFRQAPGQERESVSCAYPQGSQFYRDSVKGRFTFASDSAKNTVYLQMTDLKPEDTATYYCAATAAAYCSGFKQDGDHWGKGILVTVSSAHHSEDPS

SEQ ID NO: 51 - protein, 136 aa, Lama pacos
  PEPTIDE (9)..(127): Clone JGR-A5 Binds Nannochloropsis sp. OZ-1
AAQVQLVESGGALVQPGGSLRLSCAASGFTFSNAYMFWVRQAPGKGLEWVSTIRPAGGLTTYATSAKGRFTASRDSAKNALYLQMDSLKPEDTALYFCARYTRDVNIALIPMSPNFWGQGTQVTVSSEPKTPKPQP

SEQ ID NO: 52 - DNA, 413 bp, Lama pacos
  misc_feature: Clone JGQ-F7
ggccgctcaggtgcagctcgtggagtcggggggaggctcggtgcagcctggggggtctctgagactctcctgtgcagcctctggattcaccttcagtagctatgccttgggctgggcccgccaggttccagggaaggggctccagtgggtgtccggtgtttatagtaatggtaacacatactatgcagactccgtgaagggccgcttcaccatctccagagacaacgccaagaacacggtgtatctgcaaatggacagcctgaaaccggaggacacggccgtgtatcactgtgcggtagggggacgcggggcctgtacatactctgatggtagtctgcactgcccgaatgaatactggggccaggggaagcaggtcaccgtctcctcagcgcaccacagcgaagacccctcgg

SEQ ID NO: 53 - DNA, 368 bp, Lama pacos
  misc_feature: Clone JGQ-A7
ggccgctcaggtgcagctcgtggagtccggagggtccctgagactctcctgtgcagcctctagaagtttattcagtggcaatactatgggctggtatcgccaggctcctgggaacgagcgcgaagtggtcgctcgcatcactcctgacggaagacgaaactatgcagactccgtgaagggccgattcaccatctccagagacaacgccaagaacacgatgtatctggagatgaacaatctgaaagttgacgacacagccacctattactgtaattcagtcgcggcggtacttcgtcgtacggcctcctggggccaggggacccaagtcaccgtctcctcagaacccaagacaccaaaaccacaaccgg

SEQ ID NO: 54 - DNA, 386 bp, Lama pacos
  misc_feature: Clone JGQ-D9
ggccgctcagttgcagctcgtggagacggggggaggcttggctcagcctggggggtctctgagactctcctgtgccgcctctggaagcatctcccggccctatgccatgggctggtaccgccagattccagggaagcagcgcgagtgggtcgcccatattaatcgtgctggcagtaggacaaactatacagactccgtgaagggccgattcaccatttccagagacaataccaagaacacggtgtatttggaaatgaacagcctaaaacctgaggacacggccgtgtactactgtaatgctgtaccacggtacgggagagactactggggccaggggacccaggtcaccgtctcctcagaacccaagacaccaaaaccacaaccgg

SEQ ID NO: 55 - DNA, 389 bp, Lama pacos
  misc_feature: Clone JGR-H5
ggccgctcagttgcagctcgtggagacaggaggaggcttggtgcagactggggggtctctgagactctcctgtgcagctaatagaagcatcttcggtatcagtgccatggaatggtaccgccaggctccagggaaggatcgcgagttggtcgcacgtattaccagtggtggtagcacaaactatgatgactccgtgaagggccgattcaccatctccagagacaacgccaagaacacggtgtatctgcaaatgaacagccttaaacctgaggacacaggcgtctatttctgtaattcgcggaacggtagaaactggtcacggggttcctggggccaggggacccaggtcaccgtctcctcggaacccaagacaccaaaaccacaaccgg

SEQ ID NO: 56 - DNA, 371 bp, Lama pacos
  misc_feature: JGQ-H9
ggccgctcaggtgcagctcgtggagacggggggaggctcggtgcaggctggggggtctctgagactctcctgtgtagcctctcaaacgatctacacgatcatggcctggtaccgccaggctccagggaaagagcgcgagttggtcgcaagtgttctccgtgatggacgcacaaaccacgcagcctccgtgaaagggcgattcaccatctccagagacaacgccaagaacacgatatatctgcaaatgaacagtctgaaacctgaagacacagccatctattactgtaatgtgggcggcttcaccagcagctggggccaggggacccgggtcaccgtctcctcagaacccaagacaccaaaaccacaaccgg

SEQ ID NO: 57 - DNA, 422 bp, Lama pacos
  misc_feature: Clone JGR-G5
ggccgctcaggtgcagctcgtggagacggggggaggcttggtacaggctggggggtctctgagactctcctgtgcagcctctggaaccatcttcaatggcaatcgtatcaatgccatgggctggtaccgccagattccagggaaggagcgcgacttggtcgcaactatcactgttgatggtaaagtaaacctgggaaaccttgtgaagggccgattcaccatctccgaagacgacgctcgcaatacggtgtatctgcaaatgaaccgcctgaaacctgaggacacagccgtctattactgtactgcaggaaaactgacttcgagcggaggtgtggactactacagtccctcgaatgactactggggccaggggacccaggtcaccgtctcctcggaacccaagacaccaaaaccacaaccgg

SEQ ID NO: 58 - DNA, 377 bp, Lama pacos
  misc_feature: Clone JGR-H9
ggccgctcagttgcagctcgtggagtccggtggaggcttggtgccggctggggggtctctgagactctcctgtgcggcctctggaagcatcttcaatggcaatgtcatggcctggtaccgccgaactcctggaaatcagcgcaacatggtcgccgctattactagtagtggtagtacaacttatccacactccgtgaagggccgattcaccatctccagagacaacgccgagaacacgctgtacctacaaatgaacagcctgaaacccgaggacacagccgtctattactgcaatacactgcaaccactcaactactggggccaggggacccaggtcaccgtctcctcagaacccaagacaccaaaaccacaaccgg

SEQ ID NO: 59 - DNA, 380 bp, Lama pacos
  misc_feature: Clone JGR-G2
ggccgctcagttgcagctcgtggagacggggggaggcttggtgcaggctggggggtctctgagactctcctgtttagcctctggaagcatcgtcaatagtcagaccgtggcctggtaccgccaggctccaggaaagtcgcgcgagttcgtcgcacacattactagtcaaggcttggccgggtatgccagctccgtgagaggccgcttcaccatctccagaaacacaggcaagaacacggcgtatctacaaatgaacagtctacaacctgaggacacagccgtctattattgtactgcggacgtgagggggtacaggtactggggccaggggacccaggtcaccgtctcctcagaacccaagacaccaaaaccacaaccgg

SEQ ID NO: 60 - DNA, 398 bp, Lama pacos
  misc_feature: Clone JGR-G11
ggccgctcaggtgcagctcgtggagacggggggaggcttggtgcaggctggggggtctctgagactctcctgtgcagcctctggaagcatcagcgagatctatcgcatgggctggtaccgccaggctccagggaagcagcgcgaattggtcgcggctatcacttcccctggtaatactaactacccagactcccttaagggccgattcgccatctccagggattacgccaagaacacggggtatctacaaatggatcgcctggaacctgaggacacagctgtttattactgtaaagcaaacctactacagcgtgcgccgcgcaagtatctcgaaatttggggtcagggcaccctggtcactgtctcctcagaacccaagacaccaaaaccacaaccgg

SEQ ID NO: 61 - DNA, 389 bp, Lama pacos
  misc_feature: Clone JGQ-C12
ggccgctcaggtgcagctcgtggagtcgggcggaggcttggtccgggctggggggtctctgaagctcacctgtgcagtctctggagatatcttcagtatcaaagccatggggtggtaccgccgggctccagggaagaagcgcgagtcggtcgcggcgattagtgcggctggtaatacattgtatgcagattctgtgaagggccgattcaccatttccagagacaatgatgccaacacgctgtttctgcaaatgaacagtctgaaacccgaggacacggccatgtattactgtgcggccgaaattaataatagcgactccctaaatcaagggggccaggggacccaggtcaccgtctcctcagaacccaagacaccaaaaccacaaccgg

SEQ ID NO: 62 - DNA, 407 bp, Lama pacos
  misc_feature: Clone JGR-H6
ggccgctcaggtgcagctcgtggagtcgggtggaggattggtgcaggccgggggctctctgagactctcctgcgcaggctctggacgctccttcagcgccgctgtcatgggctggttccgccaggcgccagggaaggagcgagaattcgtagcagcacttagacaaattattggtagcacacactatgcagactccgtgaagggccgattcaccatctccagagacaacgccaagaacatgttgtatctcgacatgaacagcctgaaacctacggacacggccgcgtattactgcacagcgggacctccgactatgctggacgtttctaccgaccgggagtatgacacctggggtcaggggactcaggtcgccgtctcctcagcgcaccacagcgaagacccctcgg

SEQ ID NO: 63 - DNA, 404 bp, Lama pacos
  misc_feature: Clone JGR-H8
ggccgctcaggtgcagctcgtggagacgggagggttggtgcagactggaggctccctgagactctcctgttcagcctctggacgcaccttcagtcactataccatgggctggttccgccaggctccagggaaggagcgtgagtttgtagcaactattagtcggagtggtggtaagacagcctatgcagactccgtgaagggccgattcaccatctccagagacaacgccaagaacatggtgtttctgcaaatgaccagcctgaaatctgaggacacggccgtttattactgtgcagcctcccgactaactggcccccaacctctccatgataagtatgcctattggggccaggggacccaggtcaccgtctcctcagaacccaagacaccaaaaccacaaccgg

SEQ ID NO: 64 - DNA, 383 bp, Lama pacos
  misc_feature: JGQ-B3
ggccgctcaggtgcagctcgtggagtcggggggaggcttggtgcagactggggggtctctgagactctcctgtgtaggctctggaataagtttgactaaggatatgatggcctggtaccggcaggctccaggaaagcagcgcgagttagtcgcaactattccggtggtgggcggtggtaggcctacctatgaagactccgtgaagggccgattcaccatctcccgagacaacgccaagaacacggtgtatttggaaatgaacagcctgaaacctgaagatacaggcgactactattgtcaacggccgagcgcctggcttgcctggggccaggggacgcaggtcaccgtctcctcagaacccaagacaccaaaaccacaaccgg

SEQ ID NO: 65 - DNA, 401 bp, Lama pacos
  misc_feature: Clone JGR-E5
ggccgctcagttgcagctcgtggagacggggggaggaacggtgcaggccggggggtctctgagactctcctgtgaagcctctggattccgtttcgctgattatgccataggctggttccgccaggccccaggccaggagcgtgagtcggtgtcatgtgcttatccacaaggcagtcaattttatcgagactccgtgaagggccgattcacgttcgccagtgacagcgccaagaacacggtgtatctgcaaatgacagatctgaaacctgaggacacggctacttattactgtgcggccaccgccgcagcctactgttcaggcttcaagcaggacggagatcactggggcaaagggatcctggtcaccgtctcctcggcgcaccacagcgaagacccctcgg

SEQ ID NO: 66 - DNA, 410 bp, Lama pacos
  misc_feature: Clone JGR-A5
ggccgctcaggtgcagctcgtggagtcggggggagccttggtgcagcctggggggtctctgagactctcctgtgcagcctctggattcaccttcagtaacgcctatatgttttgggtccgtcaggctccagggaaggggctcgagtgggtctcaaccattaggcctgctggtggtctcacaacctatgcaacatccgcgaagggccgattcaccgcctccagagacagcgccaagaacgcgctctatctgcaaatggacagcctgaaacctgaggacacggccctgtatttttgtgcgagatacacccgagacgtgaatattgcactgataccgatgtctcccaatttctggggccaggggacccaggtcaccgtctcctccgaacccaagacaccaaaaccacaaccgg

SEQ ID NO: 67 - protein, 129 aa, Lama pacos
  PEPTIDE (9)..(120): Clone JGT-D4 Binds Thalassiosira pseudonana CMMP 1335
AAQLQLVETGGGLVRAGGSLKLTCAASGDIFSIKAMGWYRRAPGKKRESVAAISAAGDTLYADSVKGRFTISRDNDANTLFLQMNSLKPEDTAMYYCAAEINNSDSLNQGGQGTQVTVSSEPKTPKPQP

SEQ ID NO: 68 - protein, 134 aa, Lama pacos
  PEPTIDE (9)..(126): Clone JGS-G10 Binds Thalassiosira pseudonana CMMP 1335
AAQVQLVETGGGLVQAGGSLRLSCAVSGRTFSSLAMGWFRQAPGKEREFVAAISWSGDSTHYADSMKGRFTISRDNAKNTVYLQMDSLKPEDTAVYYCAADRTYHSGSPYWGAEYWGQGTQVTVSSAHHSEDPS

SEQ ID NO: 69 - protein, 128 aa, Lama pacos
  PEPTIDE (9)..(119): Clone JGT-H4 Binds Thalassiosira pseudonana CMMP 1335
AAQVQLVESGGGSVQAGESLRLSCVVSGTDFSSHHGGWARQGPGNTRTFFAAISSGGRTNYTSSVKGRFIISRDNAKNTMYLQMNNLKPEDTGLYVCNIRRKSYLSGDWGQGTQVTVASEPKTPKPQP

SEQ ID NO: 70 - DNA, 389 bp, Lama pacos
  misc_feature: Clone JGT-D4
ggccgctcagttgcagctcgtggagacggggggaggcttggtccgggctggggggtctctgaagctcacctgtgcagcctctggagatatcttcagtatcaaagccatggggtggtaccgccgggctccagggaagaagcgcgagtcggtcgcggcgattagtgcggctggtgatacattgtatgcagattctgtgaagggccgattcaccatttccagagacaatgatgccaacacgctgtttctgcaaatgaacagtctgaaacccgaggacacggccatgtattactgtgcggccgaaattaataatagcgactccctaaatcaagggggccaggggacccaggtcaccgtctcctcagaacccaagacaccaaaaccacaaccgg

SEQ ID NO: 71 - DNA, 404 bp, Lama pacos
  misc_feature: Clone JGS-G10
ggccgctcaggtgcagctcgtggagacggggggaggattggtgcaggctgggggctctctgagactctcctgtgcagtctctggacgcaccttcagtagcttggccatgggctggttccgccaggctccagggaaggagcgtgagtttgtagcggctattagctggagtggtgatagcacacactatgcagactccatgaagggccgattcaccatctccagagacaacgccaagaacacggtgtatctgcaaatggacagcctgaaacctgaggacacggccgtttattactgtgcagcagatcgaacataccatagtggtagtccatattggggggccgagtactggggccaggggacccaggtcaccgtctcctcagcgcaccacagcgaagacccctcgg

SEQ ID NO: 72 - DNA, 386 bp, Lama pacos
  misc_feature: Clone JGT-H4
ggccgctcaggtgcagctcgtggagtcggggggagggtcggtgcaggcgggggagtctctgagactctcctgtgtagtctctggtaccgacttcagtagtcatcacgggggttgggcccgccagggtccaggaaatacgcgcacattcttcgcagcgattagtagtggcggtcgtacaaattatacaagctccgtgaagggccgattcatcatctccagagacaacgccaagaacacgatgtatttgcagatgaacaacctgaaacctgaggacacgggtctttacgtctgtaatatacgtaggaagtcgtacttgagtggcgactggggccaggggacccaggtcaccgtcgcctcagaacccaagacaccaaaaccacaaccgg

SEQ ID NO: 73 - protein, 544 aa, Lama Pacos
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLHMASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKAAAQVQLVETGGGLVQAGGSLRLSCVVSGRNNSGIGMGWFRQAPGKEREFVAAIGWGGSSTIYADSVKGRFTVSRDNAKNTVYLQMVSLKPDDTAVYVCAARRRALTPISLAAGFEYWGQGTQVIVSAEPKTPKPQPARQGAPVPYPDPLEPR

SEQ ID NO: 74 - protein, 532 aa, Lama Pacos
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLVPVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAAAQLQLVETGGGLVQAGGSLRLSCAASGNIFRVRAMAWYRQAPGKQREWVAVLGSRGGTNYADSVKGRFTISRDNAANTIFLQMNSLKPEDTAVYYCNHYPPEKRDYWGQGTQVTVSSEPKTPKPQPARQGAPVPYPDPLEPR